(19) United States
(12) Patent Application Publication
(10) Pub. No.: US 2025/0257069 A1
(43) Pub. Date: Aug. 14, 2025

(54) JAK INHIBITOR ANALOGS, FORMULATIONS, AND USES THEREOF

(71) Applicant: THE REGENTS OF THE UNIVERSITY OF MICHIGAN, Ann Arbor, MI (US)

(72) Inventors: Zhongwei Liu, Ann Arbor, MI (US); Lu Wang, Ann Arbor, MI (US); Yingzi Bu, Ann Arbor, MI (US); Mohamed Dit Mady Traore, Ann Arbor, MI (US); Duxin Sun, Ann Arbor, MI (US)

(21) Appl. No.: 18/856,839

(22) PCT Filed: Apr. 14, 2023
(86) PCT No.: PCT/US23/18658

§ 371 Date: Oct. 14, 2024

Related U.S. Application Data
(60) Provisional application No. 63/331,463, filed on Apr. 15, 2022.

(51) Int. Cl. C07D 487/04; A61K 31/655; A61K 47/54; A61P 1/04; A61P 29/00; A61P 35/00; A61P 37/02; C07D 239/48; C07D 498/08

(52) U.S. Cl. CPC C07D 487/04; A61K 31/655; A61K 47/542; A61K 47/545; A61P 1/04; A61P 29/00; A61P 35/00; A61P 37/02; C07D 239/48; C07D 498/08

(57) ABSTRACT

The present disclosure provides JAK inhibitor analogs, and compositions and methods thereof for treating diseases or disorders (e.g., inflammatory bowel disease and ulcerative colitis).

### CROSS-REFERENCE TO RELATED APPLICATIONS

This application claims the benefit of U.S. Provisional Application No. 63/331,463, filed Apr. 15, 2022, the content of which is herein incorporated by reference in its entirety.

### FIELD

The present disclosure provides JAK inhibitor analogs, and compositions and methods thereof for treating diseases or disorders (e.g., inflammatory bowel disease and ulcerative colitis).

### BACKGROUND

Inflammatory Bowel Disease (IBD) affects more than 6.8 million patients in the world and 1-2 million patients in the US. Ulcerative Colitis (UC) accounts for ⅔ of IBD cases while Crohn's disease accounts for the remaining third of IBD cases. UC usually starts from the rectum and extends proximally to the colon where inflammation is restricted to the innermost layer of the intestine (mucosal) resulting in ulceration and bloody diarrhea. In addition, UC patients have up to 18% higher risk of developing colon cancer depending on the severity and the duration of the disease.

Current treatment options have various limitations. Anti-inflammatory treatments using controlled release formulations of 5-aminosalicylates and corticosteroids only achieve limited efficacy in reducing; the symptoms, Anti-TNF antibodies treatment is effective but requires life-long injections. Immune system suppressors such as azathioprine and cyclosporin have limited efficacy and present serious side effects after long-term use.

Inhibition of the Janus Kinases (JAK1, JAK2, JAK3 and TYK2) has recently emerged as a therapeutic approach for the treatment of UC. Several orally bioavailable small molecule JAK inhibitors, e.g., tofacitinib, have been developed and approved for the treatment of IBD and rheumatoid arthritis. However, tofacitinib, and all other JAK inhibitors, have black box warnings for serious side effects, which include: high rate of major adverse cardiovascular events (MACE) (cardiovascular death, myocardial infarction, stroke), arterial and venous thrombosis and pulmonary embolism, malignancies of lymphomas and lung cancers, and increased risk of serious infections leading to death.

### SUMMARY

In one aspect, disclosed herein is a Janus kinase (JAK) inhibitor analog, or a pharmaceutically acceptable salt thereof, wherein the JAK inhibitor analog has the structure:

A-L-B
wherein:
A is a JAK inhibitor moiety;
L is a cleavable linker; and
B is a prodrug moiety.

In some embodiments, the JAK inhibitor moiety is derived from abrocitinib, baricitinib, cerdulatinib, delgocitinib, deucravacitinib, fedratinib, filgotinib, gandotinib, lestaurtinib, momelotinib, oclacitinib, pacritinib, peficitinib, ruxolitinib, tofacitinib, or upadacitinib. In some embodiments, the JAK inhibitor moiety comprises a benzimidazole moiety, a pyrrolopyrimidine moiety, or a biaryl meta-pyrimidine moiety.

In some embodiments, the cleavable linker comprises at least one selectively cleavable group or bond. In some embodiments, the selectively cleavable group or bond is enzymatically cleavable. In some embodiments, the cleavable linker comprises an azo group.

In some embodiments, L comprises

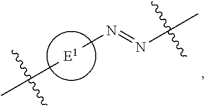

wherein E¹ is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, amino-C₁-C₆-alkyl, or —COO—R^{1a}; and R^{1a} is hydrogen or C₁-C₆ alkyl. In some embodiments, E¹ is a C₄-C₁₀ arylene or C₄-C₁₀ heteroarylene, optionally substituted with 1 or 2 substituents independently selected from C₁-C₆ alkyl, amino, C₁-C₆-alkoxy, hydroxy, hydroxy-C₁-C₆-alkyl, amino-C₁-C₆-alkyl, or —COO—R^{1a}.

In some embodiments, L comprises

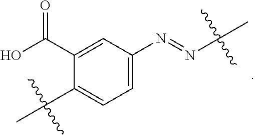 [SMILES: */N=N/c1ccc(*)c(C(=O)O)c1]

In some embodiments, L further comprises a combination of one or more groups selected from —CH₂—, —O—, —NR^{1b}—, arylene and heteroarylene and R^{1b} is hydrogen or C₁-C₆ alkyl. In some embodiments, L further comprises

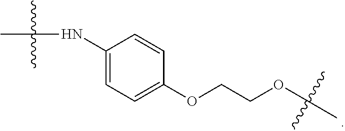 [SMILES: *Nc1ccc(OCCO*)cc1]

In some embodiments, B comprise

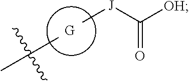

wherein G is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, or amino-C₁-C₆-alkyl; and J is a bond or a linker comprising a combination of one or more groups selected from —C(R^{1c})₂—, —CH═CH—, —C≡C—, —O—, —NR^{1c}—, —S—, —C(O)—, —C(NR^{1c})—, —S(O)—, and —S(O)₂—, wherein each RIG is independently selected from hydrogen, C₁-C₆ alkyl, C₂-C₆ alkenyl, or C₂-C₆ alknyl. In some embodiments, J is a linker comprising a combination of one or more groups selected from —C(R^{1c})₂—, —NR^{1c}—, and —C(O)—, wherein each R^{1c} is independently selected from hydrogen and C₁-C₆ alkyl. In some embodiments, J comprises

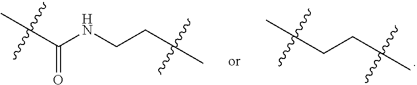

In some embodiments. J is a bond.

In some embodiments B comprises

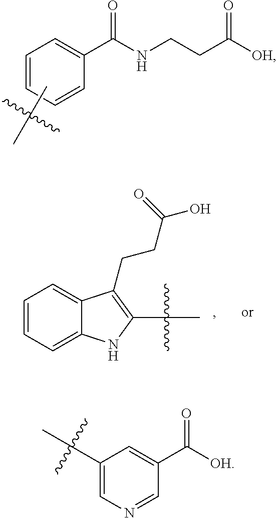

In some embodiments, JAK inhibitor analog; is a compound of formula (I):

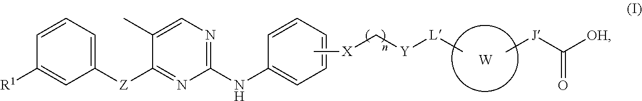

or a pharmaceutically acceptable salt thereof, wherein:
Z is NR^{a}, wherein R^{a} is H or C₁-C₆ alkyl;
R¹ is alkyl or SO₂—R², wherein R² is selected from C₁-C₆ alkyl, C₃-C₉ cycloalkyl, C₃-C₉ heterocycle, and N(R^{b})₂, and wherein each R^{b} is independently selected from hydrogen, C₁-C₆ alkyl, C₃-C₉ cycloalkyl, and C₃-C₉ heterocycle, or both R^{b} are taken together with the nitrogen atom to which they are attached to form an optionally substituted 5- or 6-membered ring;
X is O, SO₂, or CH₂;
Y is NH, O, or CH₂;
W is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, or amino-C₁-C₆-alkyl;
J′ is a bond or a linker comprising a combination of one or more groups selected from —C(R^{c})₂—, —CH═CH—, —C≡C—, —O—, —NR^{c}—, —S—, —C(O)—, —C(NR^{c})—, —S(O)—, and —S(O)₂—, wherein each R^{c} is independently selected from hydrogen, C₁-C₆ alkyl, C₂-C₆ alkenyl, or C₂-C₆ alkynyl;
n is 1, 2, 3, 4, 5, or 6; and
L′ is cleavable linker.

In some embodiments, J′ is a linker comprising a combination of one or more groups selected from —C(R^{c})₂—, —NR^{c}—, and —C(O)—, wherein each R^{c} is independently selected from hydrogen and C₁-C₆ alkyl. In some embodiments, J′ comprises

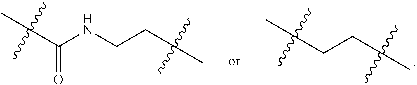

In some embodiments, J′ is a bond.

In some embodiments, the JAK inhibitor analog is a compound of formula (Ia):

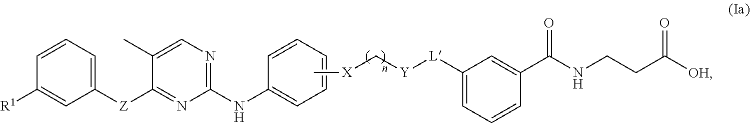

or a pharmaceutically acceptable salt thereof.

In some embodiments, Z is NH. In some embodiments, R¹ is —SO₂—N(R^{b})₂. In some embodiments, one R^{b} is hydrogen and the other is C₁-C₆ alkyl. In some embodiments, X and Y are O. In some embodiments, n is 1, 2, or 3.

In some embodiments, L′ comprises

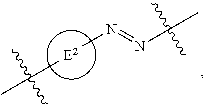

E² is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, amino-C₁-C₆-alkyl, or —COO—R^{d}; and R^{d} is hydrogen or C₁-C₆ alkyl. In some embodiments, E² is a C₄-C₁₀ arylene or C₄-C₁₀ heteroarylene, optionally substituted with 1 or 2 substituents independently selected from C₁-C₆ alkyl, amino, C₁-C₆-alkoxy, hydroxy, hydroxy-C₁-C₆-alkyl, amino-C₁-C₆-alkyl, or —COO—R^{d}.

In some embodiments, L′ comprises

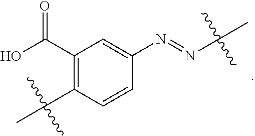 [SMILES: */N=N/c1ccc(*)c(C(=O)O)c1]

In some embodiments, L′ further comprises a combination of one or more groups selected from —CH₂—, —O—, —NR^{c}—, arylene and heteroarylene.

In some embodiments, L′ further comprises

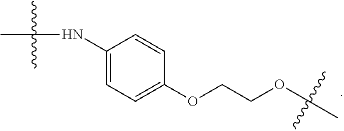 [SMILES: *Nc1ccc(OCCO*)cc1]

In some embodiments, the compound is:

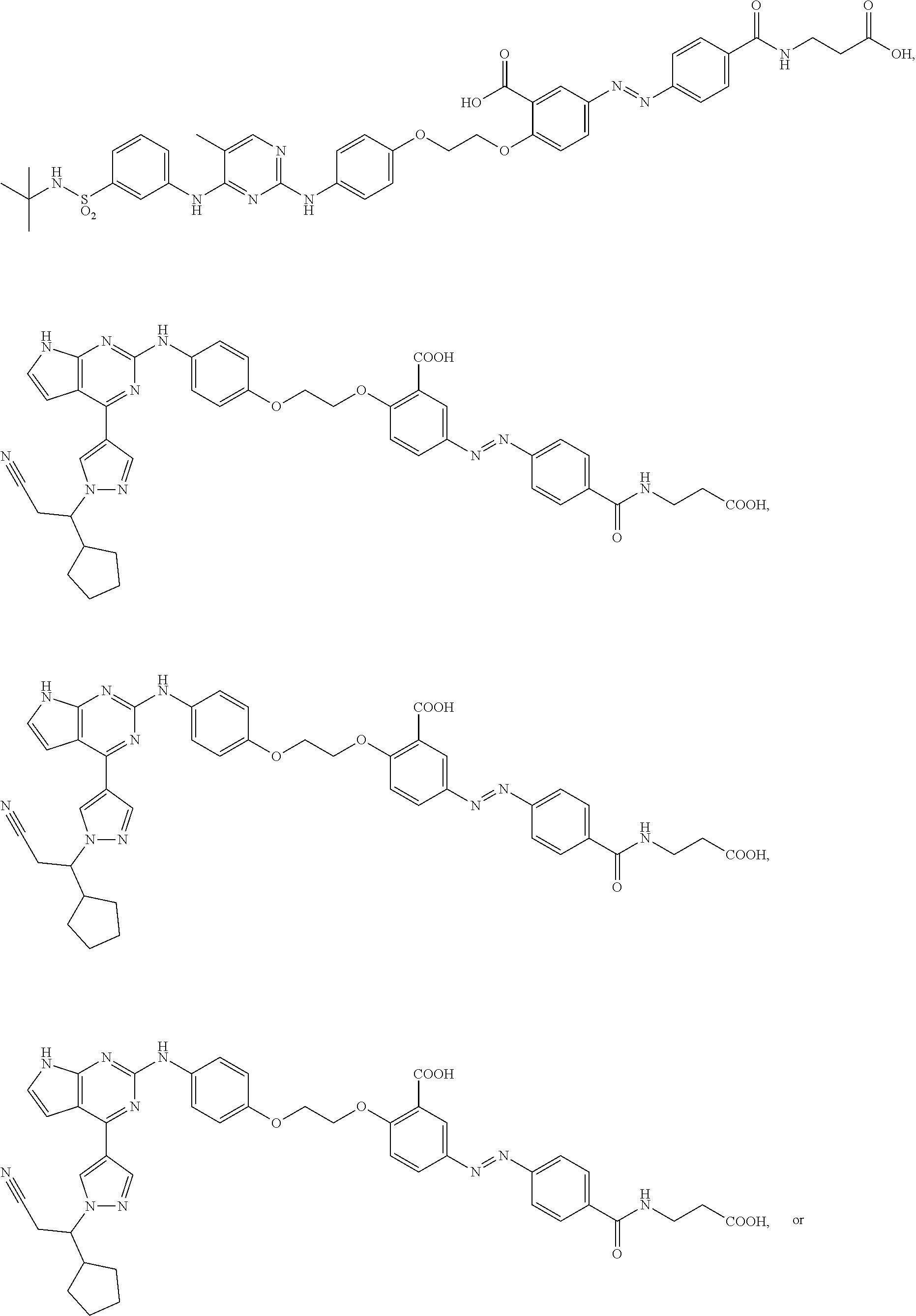 [SMILES: Cc1cnc(Nc2ccc(OCCOc3ccc(/N=N/c4ccc(C(=O)NCCC(=O)O)cc4)cc3C(=O)O)cc2)nc1Nc1cccc(S(=O)(=O)NC(C)(C)C)c1 ; N#CCC(C1CCCC1)n1cc(-c2nc(Nc3ccc(OCCOc4ccc(/N=N/c5ccc(C(=O)NCCC(=O)O)cc5)cc4C(=O)O)cc3)nc3[nH]ccc23)cn1 ; N#CCC(C1CCCC1)n1cc(-c2nc(Nc3ccc(OCCOc4ccc(/N=N/c5ccc(C(=O)NCCC(=O)O)cc5)cc4C(=O)O)cc3)nc3[nH]ccc23)cn1 ; N#CCC(C1CCCC1)n1cc(-c2nc(Nc3ccc(OCCOc4ccc(/N=N/c5ccc(C(=O)NCCC(=O)O)cc5)cc4C(=O)O)cc3)nc3[nH]ccc23)cn1 ; "or"]

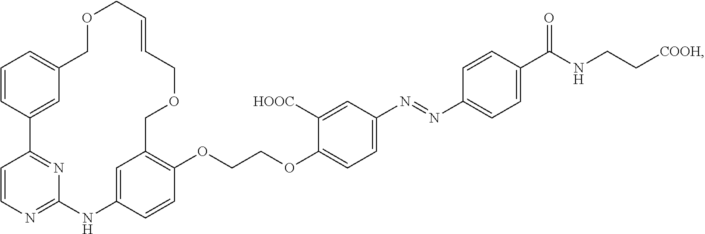 [SMILES: O=C(O)CCNC(=O)c1ccc(/N=N/c2ccc(OCCOc3ccc4cc3COC/C=C/COCc3cccc(c3)-c3ccnc(n3)N4)c(C(=O)O)c2)cc1]

or a pharmaceutically acceptable salt thereof.

In another aspect, disclosed herein is a pharmaceutical composition comprising an effective amount of a JAK inhibitor analog disclosed herein (e.g., a compound of formula A-L-B, or a compound of formula (I) or (Ia)), or a pharmaceutically acceptable salt thereof, and a pharmaceutically acceptable carrier.

In another aspect, disclosed herein is a method of treating or preventing a disease or disorder comprising administering an effective amount of a JAK inhibitor analog disclosed herein (e.g., a compound of formula A-L-B, or a compound of formula (I) or (Ia)), or a pharmaceutically acceptable salt thereof, or a pharmaceutical composition comprising a JAK inhibitor analog disclosed herein, or a pharmaceutically acceptable salt thereof, to a subject in need thereof.

In some embodiments, the disease or disorder is cancer, an autoimmune disease, or an inflammatory disease. In some embodiments, the disease or disorder is a gastrointestinal inflammatory disease or disorder. In some embodiments, the disease or disorder is inflammatory bowel disease. In some embodiments, the inflammatory bowel disease is ulcerative colitis or Crohn's disease. In some embodiments, the disease or disorder is cancer. In some embodiments, the subject has cancer, has had cancer, is predisposed to cancer, or has a family history of cancer. In some embodiments, the JAK inhibitor analog, or a pharmaceutically acceptable salt or composition thereof is administered orally.

In another aspect, disclosed herein is a compound of formula (II):

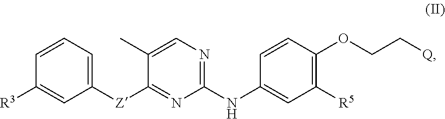

or a pharmaceutically acceptable salt thereof, wherein:
Q is

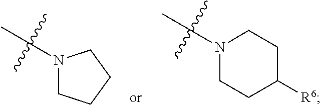

Z′ is NR^{c}, wherein R^{c} is H or C₁-C₆ alkyl;
R³ is alkyl or SO₂—R⁴, wherein R⁴ is selected from C₁-C₆ alkyl, C₃-C₉ cycloalkyl, C₃-C₉ heterocycle, and N(R^{d})₂, and wherein each R^{d} is independently selected from hydrogen, C₁-C₆ alkyl, C₃-C₉ cycloalkyl, and C₃-C₉ heterocycle, or both R^{d} are taken together with the nitrogen atom to which they are attached to form an optionally substituted 5- or 6-membered ring;
R⁵ is hydrogen, —CH₂—OCH₃ or —CH₂—(OCH₂CH₂)—OCH₃; and
R⁶ is —OCH₃ or —OCH₂CH₂—OCH₃.

In some embodiments, Z′ is NH. In some embodiments, R³ is SO₂—N(R^{d})₂. In some embodiments, one R^{d} is hydrogen and one R^{d} is C₁-C₆ alkyl. In some embodiments, Q is

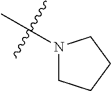 [SMILES: *N1CCCC1]

and R⁵ is —CH₂—OCH₃ or —CH₂—(OCH₂CH₂)—OCH₃. In some embodiments, Q is

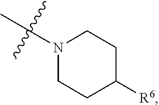

R⁵ is hydrogen, and R⁶ is —OCH₃ or —OCH₂CH₂—OCH₃. In some embodiments, Q is

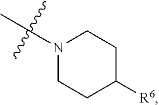

R⁵ is —CH₂—OCH₃, and R⁶ is —OCH₃ or —OCH₂CH₂—OCH₃. In some embodiments, Q is

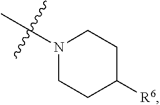

R⁵ is —CH₂—(OCH₂CH₂)—OCH₃, and R⁶ is —OCH₃— or —OCH₂OCH₂—OCH₃.

In another aspect, disclosed herein is a pharmaceutical composition comprising an effective amount of a compound of formula (II), or a pharmaceutically acceptable salt thereof, and a pharmaceutically acceptable carrier.

In another aspect, disclosed herein is a method of treating or preventing a disease or disorder comprising administering an effective amount of a compound of formula (II), or a pharmaceutically acceptable salt thereof, or a pharmaceutical composition comprising a compound of formula (II), or a pharmaceutically acceptable salt thereof, to a subject in need thereof.

Other aspects and embodiments of the disclosure will be apparent in light of the following detailed description and accompanying figures.

### BRIEF DESCRIPTION OF THE DRAWINGS

FIG. 1A is a schematic of the pharmacokinetics of an exemplary GI locally-activating JAK inhibitor. FIG. 1B is a schematic of the design of an exemplary GI locally-activating JAK inhibitor. FIG. 1C shows the co-crystal structure of JAK2 with fedratinib (PDB 6VNE).

FIG. 2 shows structures of MMT3-72 and its 5 metabolites: MMT3-72-M1, MMT3-72-M2, MMT3-72-M3, MMT3-72-M4, and MMT3-72-M5.

FIG. 3 are graphs of the inhibition of different isoform of JAKs by MMT3-72 and active metabolite MMT3-72-M2. Inhibition of JAK activity by MMT3-72 and MMT3-72-M2 (0.01-10,000 nM) was measured using Kinase-Glo Max assay against purified enzymes JAK1, JAK2, JAK3, TYK2. The IC₅₀ of compounds to inhibit different JAK isoforms were calculated using Prism 8.

FIGS. 4A-4C are graphs of the concentration of MMT3-72 and MMT3-72-M2 in GI content, GI tissues, and plasma. FIG. 4A is a graph of the MMT3-72 concentrations in plasma, colon tissue, small intestine tissue, colon content and small intestine content at 0.5 h, 2 h, 4 h, 12 h and 24 h. FIG. 4B is a graph of the MMT3-72-M2 concentrations in plasma, colon tissue, and small intestine tissue at 0.5 h, 2 h, 4 h, 12 h and 24 h. The dotted line showed IC₅₀ of MMT3-72-M2 in inhibiting JAK1, JAK2, JAK3 and TYK2, respectively. FIG. 4C is a graph of the MMT3-72-M2 concentrations in colon content, small intestine content, and stomach content at 0.5 h, 2 h, 4 h, 12 h and 24 h.

FIGS. 5A-5H are graphs of the in vivo efficacy MMT3-72 in comparison with tofacitinib for UC treatment. FIG. 5A is a graph showing the improvement of UC DAI score after treatment of MMT3-72 and tofacitinib (1, 5 mg/kg). FIG. 5B is a graph showing the recovery of colon length from DSS-induced colitis after treatment of MMT3-72 and tofacitinib (1, 5 mg/kg). FIG. 5C is a graph showing the percentage of mice with severe colitis and gross bleeding on day 5 after treatment of MMT3-72 and tofacitinib (1, 5 ng/kg). FIG. 5D is a graph showing the percentage of mice with moderate colitis on day 5 after treatment of MMT3-72 and tofacitinib (1, 5 mg/kg). FIG. 5E is a graph showing the improvement of UC DAI score after treatment of MMT3-72 and tofacitinib (10 mg/kg). FIG. 5F is a graph showing the recovery of colon length after treatment of MMT3-72 and tofacitinib (10 mg/kg). FIG. 5G is a graph showing the percentage of mice developing severe colitis with gross bleeding; on day 5 after treatment of MMT3-72 and tofacitinib (10 mg/kg). FIG. 5H is a graph showing the percentage of mice developing moderate colitis on day 5 after treatment of MMT3-72 and tofacitinib (10 mg/kg).

FIG. 6 is images of H&E staining of colon tissues after treatment of MMT3-72 and tofacitinib in DSS-induced colitis model. Control was H&E staining of healthy mice colon tissue. DSS-induced colitis showed disrupted epithelium and infiltration of immune cells in colin tissues. Treatment of MMT3-72 (5, 10 mg) reduced epithelium disruption and infiltration of immune cells in colon tissues in comparison with tofacitinib (5, 10 mg/kg) in DSS-induced colitis model.

FIG. 7 shows structures of MMT3-56, MMT3-84, MMT3-83, MMT3-85, MMT3-73, MMT3-89, MMT3-79, and MMT3-90.

FIGS. 8A and 8B are graphs of the inhibition of cell growth in JAK related cell lines HEL cells (FIG. 8A) and SET-2 cells (FIG. 8B).

### DETAILED DESCRIPTION

Described herein are JAK inhibitor analogs and compositions thereof. An exemplary GI locally-activating JAK inhibitor analog maximized drug exposure to the intestinal tissue resulting in superior efficacy in UC treatment while reducing system drug exposure thereby lessening the adverse side effects of JAK inhibitors.

The inactivated, synthesized compound MMT3-72 showed minimal inhibitory activities against JAKs (JAK1, JAK2 JAK3 and TYK2) and low absorption potential to systemic circulation. However, upon activation, primarily in the colon, MMT3-72-M2 was released and showed potent inhibitory activities against JAK1/2 and TYK2. MMT3-72 accumulated in the GI lumen but not in GI tissue nor in plasma, whereas the released active metabolite MMT3-72-M2 accumulated in the colon lumen and colon tissue with minimal exposure in the plasma. MMT3-72 (PO, 5, 10 mg/kg) achieved superior efficacy to tofacitinib in dextran sulfate sodium (DSS)-induced colitis in mice.

Section headings as used in this section and the entire disclosure herein are merely for organizational purposes and are not intended to be limiting.

### 1. DEFINITIONS

The terms “comprise(s),” “include(s),” “having.” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular. All publications, patent applications, patents and other references mentioned herein are incorporated by reference in their entirety.

As used herein, the term “linker,” “linking group,” and “linkage” are used interchangeably to refer to a linking moiety that connects two groups and has a backbone of any suitable length. In some cases, the linker has a backbone of 20 atoms or less in length. A linker or linkage may be a covalent bond that connects two groups or a chain of any convenient length (e.g., between 1 and 20 atoms in length), for example of about 1, 2, 3, 4, 5, 6, 8, 10, 12, 14, 16, 18 or 20 carbon atoms in length, where the linker may be linear, branched, cyclic or a single atom. A linker may include, without limitations, poly(ethylene glycol), modified polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3, or 4 atoms, of the cyclic group are included in the backbone. A linker may be cleavable or non-cleavable.

As used herein, the term “moiety” is used to refer to a portion of an entity or molecule, in some cases having a particular function, structure, or structural feature.

As used herein, the terms “providing.” “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, patient may include either adults or juveniles (e.g., children) Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, “treat,” “treating,” and the like means a slowing, stopping, or reversing of progression of a disease or disorder when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito. 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched, saturated hydrocarbon chain. Representative examples of alkyl include, but are not limited to, methyl, ethyl, n-propyl, iso-propyl, n-butyl, sec-butyl, iso-butyl, tent-butyl, n-pentyl, isopentyl, neopentyl, and n-hexyl.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

The term “alkoxyalkyl,” as used herein, refers to an alkyl group, as defined herein, in which at least one hydrogen atom (e.g., one hydrogen atom) is replaced with an alkoxy group, as defined herein. Representative examples of alkoxyalkyl include, but are not limited to, methoxymethyl.

The term “amino,” as used herein, refers to an —NH₂ group. The term “alkylamino,” as used herein, refers to a group —NHR, wherein R is an alkyl group as defined herein. The term “dialkylamino,” as used herein, refers to a group —NR₂, wherein each R is independently an alkyl group as defined herein.

The term “aminoalkyl,” as used herein, refers to an alkyl group, as defined herein, in which at least one hydrogen atom (e.g., one hydrogen atom) is replaced with an amino group.

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl,” i.e., phenyl). In some embodiments, an aryl group has ten ring carbon atoms (“C₁₀ aryl,” e.g., naphthyl such as 1-naphthyl and 2-naphthyl).

As used herein, the term “arylene” refers to a divalent aryl radical.

The term “azo group,” as sued herein, refers to a group with the general formula R—N═N—R′, where R and R′ can independently be either aryl or alkyl groups.

The term “benzimidazole,” as used herein, refers to a bicyclic heteroaryl group having the following formula:

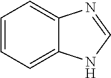

The term “biaryl meta-pyrimidine.” as used herein, refers to a group having the following structure:

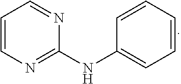 [SMILES: c1ccc(Nc2ncccn2)cc1]

The term “cycloalkyl” as used herein, refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, adamantyl, bicyclo[2.2.1]heptanyl, bicyclo[3.2.1]octanyl, and bicyclo[5.2.0]nonanyl.

As used herein, the term “cycloalkylene” refers to a divalent cycloalkyl radical.

As used herein, the term “heteroalkyl” refers to an alkyl group, as defined herein, in which one or more of the carbon atoms (and any associated hydrogen atoms) are each independently replaced with a heteroatom group such as —NH—, —O—, —S—, —S(O)—, —S(O)₂—, —OP(O)(O⁻)O—, or the like. By way of example, 1, 2, 3, 4, 5, 6, or more carbon atoms may be independently replaced with the same or different heteroatom group. A heteroalkyl group can also include one or more carbonyl moieties (i.e., wherein a carbon atom of the alkyl group is oxidized to a —C(O)— group).

As used herein, the term “heteroalkylene” refers to a divalent heteroalkyl radical.

As used herein, the term “heteroaryl” refers to an aromatic group having a single ring (monocyclic) or multiple rings (bicyclic or tricyclic), having one or more ring heteroatoms independently selected from O, N, and S. The aromatic monocyclic rings are five- or six-membered rings containing at least one heteroatom independently selected from O, N, and S (e.g., 1, 2, 3, or 4 heteroatoms independently selected from O, N, and S). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl. 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazol, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, triazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heteroarylene” refers to a divalent heteroaryl radical.

As used herein, the term “heterocyclyl” refers to a radical of a 3- to 10-membered non-aromatic ring system having ring carbon atoms and 1 to 4 ring heteroatoms wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocycyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrroiyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the term “heterocyclylene” refers to a divalent heterocyclyl radical.

As used herein, the term “hydroxy” or “hydroxyl” refers to an —OH group.

The term “hydroxyalkyl,” as used herein, refers to an alkyl group, as defined herein, in which at least one hydrogen atom (e.g., one hydrogen atom) is replaced with a hydroxy group.

As used herein, the term “pyrrolopyrimidine” refers to a bicyclic heteroaryl group having the following formula (i.e., a 7H-pyrrolo[2,3-d]pyrimidine group):

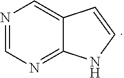

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogen atoms on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, sulfonamido, thiol, thione, thioxo, or combinations thereof.

As used herein, in chemical strictures the indication: [symbol] represents a point of attachment of one moiety to another moiety.

In some instances, the number of carbon atoms in a hydrocarbyl substituent (e.g., alkyl alkenyl) is indicated by the prefix “Cₓ-C_{y},” wherein x is the minimum and y is the maximum number of carbon atoms in the substituent. Thus, for example, “C₁-C₃alkyl” refers to an alkyl substituent containing from 1 to 3 carbon atoms.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— is intended to encompass —OCH₂—, and —C(O)NH— is intended to encompass —NHC(O)—.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. JANUS KINASE (JAK) INHIBITOR ANALOGS

In one aspect, provided herein is a Janus kinase (JAK) inhibitor analog, or a pharmaceutically acceptable salt thereof. In some embodiments, the JAK inhibitor analog has the structure:

A-L-B
wherein:
A is a JAK inhibitor moiety;
L is a cleavable linker; and
B is a prodrug moiety.

The JAK family plays a role in the cytokine-dependent regulation of proliferation and function of cells involved in immune response. Inhibitors of members of the JAI family have therapeutic efficacy in the treatment of cancer, and autoimmune and inflammatory diseases Currently, there are four known mammalian JAK family members: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2). The JAK proteins range in size from 120 to 140 kDa and comprise seven conserved JAK homology (JH) domains; one of these is a functional catalytic kinase domain, and another is a pseudokinase domain potentially serving a regulatory function and/or serving as a docking site for Signal Transducers and Activators of Transcription (STATs).

As used herein, a “JAK inhibitor moiety” refers to a moiety that inhibits at least one activity of a JAK kinase. In some embodiments, the JAK inhibitor moiety comprises a benzimidazole moiety, a pyrrolopyrimidine moiety, or a biaryl meta-pyrimidine moiety. For example, JAK inhibitor compounds having a biaryl meta-pyrimidine moiety are disclosed in WO 2007/053452, which is incorporated herein by reference.

The JAK inhibitor moiety may be derived from any known JAK inhibitor. In some embodiments, the JAK inhibitor moiety is derived from abrocitinib, baricitinib, cerdulatinib, delgocitinib, deucravacitinib, fedratinib, filgotinib, gandotinib, lestaurtinib, momelotinib, oclacitinib, pacritinib, peficitinib, ruxolitinib, tofacitinib, or upadacitinib. In some embodiments, the JAK inhibitor moiety is derived from fedratinib.

The JAK inhibitor moiety may inhibit one or more of the JAK family members. In some embodiments, the JAK inhibitor moiety decreases the kinase activity of JAK1. In some embodiments, the JAK inhibitor moiety decreases the kinase activity of JAK2. In some embodiments, the JAK inhibitor moiety decreases the kinase activity of JAK3. In some embodiments, the JAK inhibitor moiety decreases the kinase activity of TYK2.

In some embodiments, the JAK inhibitor moiety decreases the kinase activity of JAK1 and JAK2. In some embodiments, the JAK inhibitor moiety decreases the kinase activity of JAK1 and JAK3. In some embodiments, the JAK inhibitor moiety decreases the kinase activity of JAK2 and JAK3. In some embodiments, the JAK inhibitor moiety decreases the kinase activity of JAK1, JAK2, and JAK3. In some embodiments, the JAK inhibitor moiety is a pan-JAK inhibitor.

The cleavable linker includes any linker that can be selectively cleaved to produce at least two products. As such, a cleavable linker may comprise at least one selectively cleavable group or bond. A cleavable linker of the present invention is stable until it is contacted with a cleavage-inducing stimulus, e.g., an enzyme, a chemical agent, or a change in chemical conditions, which cleaves the selectively cleavable group or bond. Cleavable linkers include electrophilically cleavable linkers, nucleophilically cleavable linkers, photocleavable linkers, metal cleavable linkers, electrolytically cleavable, enzymatically cleavable linkers, linkers that are cleavable under reductive or oxidative conditions (e.g., a disulfide linker or a diazobenzene linker) and linkers that are cleavable using an acidic reagent or a basic reagent.

In some embodiments, the cleavable linker includes an enzymatically cleavable group or bond. Enzymatic reactions useful in cleaving linkers include reactions mediated by nucleases, peptidases, proteases, phosphatases, esterases, oxidases, reductases, sulfatases, etc. For example, in certain embodiments, the enzymatically cleavable linker includes, but is not limited to, β-glucuronide linkers, peptide-based linkers, and arylsulfate, disulfide, hydrazone, acetal, aminal, ester, phosphate, or azo linkers.

In some embodiments, the cleavable linker is pH sensitive. In certain embodiments, the linker comprises a low pH-labile group or bond. As used herein, a low-pH labile group or bond is a group or bond that is selectively broken under acidic conditions (pH<7). For example, in certain embodiments, the linker comprises an amine, an imine, an ester, a benzoic imine, an amino ester, a diortho ester, a polyphosphoester, a polyphosphazene, an acetal, a vinyl ether, a hydrazone, an azidomethyl-methylmaleic anhydride, a thiopropionate, a masked endosomolytic agent or a citraconyl group. In some embodiments, the cleavable bond is selected from the following: ketals that are labile in acidic environments (e.g., pH less than 7, greater than about 4) to for a diol and a ketone; acetals that are labile in acidic environments (e.g., pH less than 7, greater than about 4) to form a diol and an aldehyde; imines or iminiums that are labile in acidic environments (e.g., pH less than 7, greater than about 4) to form an amine and an aldehyde or a ketone; silicon-oxygen-carbon linkages that are labile under acidic condition; silicon-nitrogen (silazane) linkages, silicon-carbon linkages (e.g., acylsilanes, vinylsilanes, and allylsilanes); maleamates (amide bonds synthesized from maleic anhydride derivatives and amines); ortho esters; hydrazones; activated carboxylic acid derivatives (e.g. esters amides) designed to undergo acid catalyzed hydrolysis); or vinyl ethers.

In some embodiments, the linker comprises:

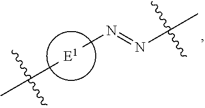

wherein E¹ is a C₄-C₁₀ cycloalkylene, C₄-C₁₀ heterocyclylene, C₄-C₁₀ arylene or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, amino-C₁-C₆-alkyl, or —COO—R^{1a}, wherein R^{1a} is hydrogen or C₁-C₆ alkyl.

In some embodiments, E¹ is a C₄-C₁₀ arylene or C₄-C₁₀ heteroarylene, optionally substituted with 1 or 2 substituents independently selected from C₁-C₆ alkyl, amino, C₁-C₆-alkoxy, hydroxy, hydroxy-C₁-C₆-alkyl, amino-C₁-C₆-alkyl, or —COO—R^{1a}, wherein R^{1a} is hydrogen or C₁-C₆ alkyl. In some embodiments, E¹ is a monocyclic arylene or heteroarylene, optionally substituted with —COO—R^{1a}, wherein R^{1a} is hydrogen or C₁-C₆ alkyl. In some embodiment, E¹ is phenylene.

In some embodiments, the linker comprises:

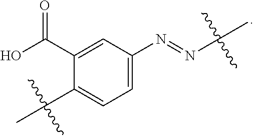 [SMILES: */N=N/c1ccc(*)c(C(=O)O)c1]

In some embodiments, the linker further comprises a combination of one or more groups selected from —C(R^{1b})₂—, —CH═CH—, —C≡C—, —O—, —NR^{1b}—, —S—, —C(O)—, —C(NR^{1b})—, —S(O)—, —S(O)₂—, arylene, heteroarylene, cycloalkylene, and heterocyclylene, wherein each R^{1b} is independently selected from hydrogen, C₁-C₆ alkyl, C₂-C₆ alkenyl, C₂-C₆ alknyl, aryl, arylalkyl, cycloalkyl, cycloalkylalkyl, heterocyclyl, heterocyclyl, heteroaryl, and heteroarylalkyl, and wherein each alkyl, alkenyl, alkynyl, arylene, heteroarylene, cycloalkylene, and heterocyclylene is independently unsubstituted or substituted with 1, 2, 3, or 4 substituents. In some embodiments, the linker further comprises a combination of one or more groups selected from —CH₂—, —O—, —NR^{1b}—, arylene and heteroarylene. In some embodiments, the linker further comprises

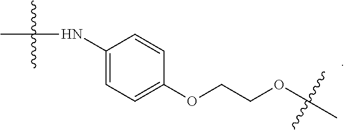 [SMILES: *Nc1ccc(OCCO*)cc1]

A prodrug moiety is a moiety that modulates the absorption, distribution, metabolization or excretion characteristics of the compound to which it is appended or linked to improve the bioavailability and/or efficacy of the compound. In some instances, the prodrug moiety renders the compound largely inactive until a transformation converts the compound to a pharmacological active form, usually as a result of removal of the prodrug moiety by an enzyme-mediated or chemical transformation.

In some embodiments, the prodrug moiety comprises

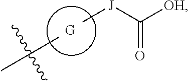

wherein G is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, or amino-C₁-C₆-alkyl, and J is a bond or a linker comprising a combination of one or more groups selected from —C(R^{1c})₂—, —CH═CH—, —C≡C—, —O—, —NR^{1c}—, —S—, —C(O)—, —C(NR^{1c})—, —S(O)—, and —S(O)₂—, wherein each R^{1c} is independently selected from hydrogen, C₁-C₆ alkyl, C₂-C₆ alkenyl, or C₂-C₆ alkynyl.

In some embodiments, C is a monocyclic arylene or heteroarylene. In some embodiments, G is phenylene. In some embodiments, G is a bicyclic arylene or heteroarylene.

In some embodiments, J is a linker comprising a combination of one or more groups selected from —CH₂— (e.g., methylene, ethylene, n-propylene, butylene, and the like), —C(O)—, and —NH—. In some embodiments, J comprises

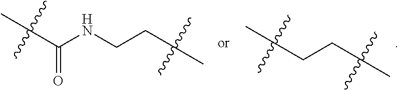

In some embodiments, J is a bond.

In some embodiments, the prodrug moiety comprises:

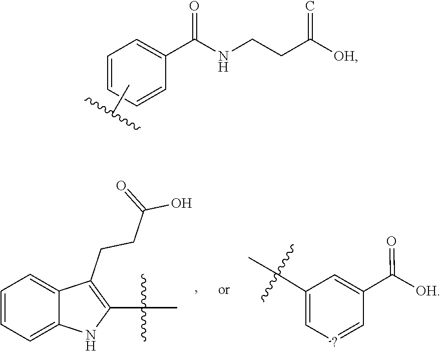

In one aspect, the JAK inhibitor analog is a compound of formula (I):

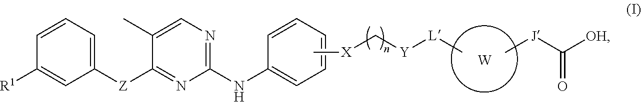

or a pharmaceutically acceptable salt thereof, wherein:
Z is NR^{a}, wherein R^{a} is H or C₁-C₆ alkyl;
R¹ is alkyl or SO₂—R², wherein R² is selected from C₁-C₆ alkyl, C₃-C₉ cycloalkyl, C₃-C₉ heterocycle, and N(R^{b})₂, and wherein each R^{b} is independently selected from hydrogen, C₁-C₆ alkyl, C₃-C₉ cycloalkyl, and C₃-C₉ heterocycle, or both R^{b} are taken together with the nitrogen atom to which they are attached to form an optionally substituted 5- or 6-membered ring;
X is O, SO₂, or CH₂;
Y is NH, O, or CH₂;
W is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, or amino-C₁-C₆-alkyl;
J′ is a bond or a linker comprising a combination of one or more groups selected from —C(R^{c})₂—, —CH═CH—, —C≡C, —O—, —NR^{c}—, —S—, —C(O)—, —C(NR^{c})—, —S(O)—, and —S(O)₂—, wherein each R^{c} is independently selected from hydrogen, C₁-C₆ alkyl, C₂-C₆ alkenyl, and C₂-C₆ alkynyl;
n is 1, 2, 3, 4, 5, or 6; and
L′ is cleavable linker.

In some embodiments, J′ comprises a combination of one or more groups selected from —CH₂— (e.g., methylene, ethylene, n-propylene, butylene, and the like), —C(O)—, and —NH—. In some embodiments, J′ comprises

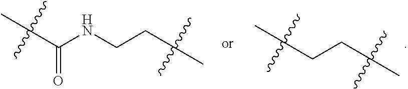

In some embodiments, p is 0.

In some embodiments, the JAK inhibitor analog is a compound of formula (Ia):

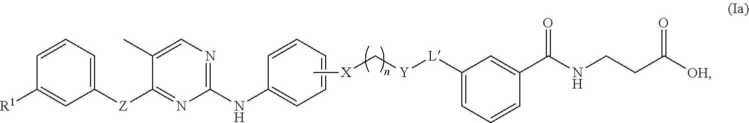

or a pharmaceutically acceptable salt thereof, wherein:
Z is NR^{a}, wherein R^{a} is H or C₁-C₆ alkyl;
R¹ is alkyl or SO₂—R², wherein R² is selected from C₁-C₆ alkyl, C₃-C₉ cycloalkyl C₃-C₉ heterocycle, and N(R^{b})₂, and wherein each R^{b} is independently selected from hydrogen, C₁-C₆ alkyl, C₃-C₉ cycloalkyl, and C₃-C₉ heterocycle, or both R^{b} are taken together with the nitrogen atom to which they are attached to form an optionally substituted 5- or 6-membered ring;
X is O, SO₂, or CH₂;
Y is NH, O, or CH₂;
n is 1, 2, 3, 4, 5, or 6; and
L′ is cleavable linker.

In some embodiments, Z is NH.

In some embodiments, R¹ is SO₂—N(R^{b})₂. In some embodiments, each R^{b} is independently C₁-C₆ alkyl. In some embodiments, one R^{b} is hydrogen and one R^{b} is C₁-C₆ alkyl.

In some embodiments, Z is NH and R² is SO₂—N(R^{b})₂.

In some embodiments, X is O. In some embodiments, Y is O. In some embodiments, X and Y are O.

In some embodiments, n is 1, 2, or 3.

In some embodiments, L′ comprises,

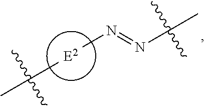

wherein E² is a C₄-C₁₀ cycloalkylene, C₄-C₁₀ to heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, amino-C₁-C₆-alkyl, or —COO—R^{d}, wherein R^{d} is hydrogen or C₁-C₆ alkyl.

In some embodiments, E² is a C₄-C₁₀ arylene or C₄-C₁₀ heteroarylene, optionally substituted with 1 or 2 substituents independently selected from C₁-C₆ alkyl, amino, C₁-C₆-alkoxy, hydroxy, hydroxy-C₁-C₆-alkyl, amino-C₁-C₆-alkyl, and —COO—R^{d}, wherein R^{d} is hydrogen or C₁-C₆ alkyl. In some embodiments, E² is a monocyclic arylene or heteroarylene, optionally substituted with —COO—R^{d}, wherein R^{d} is hydrogen or C₁-C₆ alkyl. In some embodiment, E¹ is phenylene.

In some embodiments, L′ comprises:

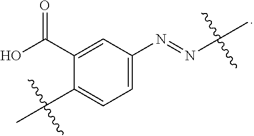 [SMILES: */N=N/c1ccc(*)c(C(=O)O)c1]

In some embodiments, L′ further comprises a combination of one or more groups selected from —C(R^{c})₂—, —CH═CH—, —C≡C—, —O—, —NR^{c}—, —S—, —C(O)—, —C(NR^{c})—, —S(O)—, —S(O)₂—, arylene, heteroarylene, cycloalkylene, and heterocyclylene, wherein each Re is independently selected from hydrogen, C₁-C₆ alkyl, C₂-C₆ alkenyl, C₂-C₆ alkynyl, aryl, arylalkyl, cycloalkyl, cycloalkylalkyl, heterocyclyl, heterocyclyl, heteroaryl, and heteroarylalkyl, and wherein each alkyl, alkenyl, alknyl, arylene, heteroarylene, cycloalkylene, and heterocyclylene is independently unsubstituted or substituted with 1, 2, 3, or 4 substituents. In some embodiments, the linker further comprises a combination of one or more groups selected from —CH₂—, —O—, —NR^{c}—, arylene and heteroarylene. In some embodiments, L′ further comprises

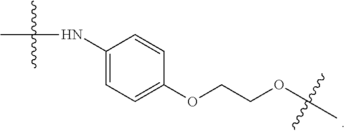 [SMILES: *Nc1ccc(OCCO*)cc1]

In some embodiments, the JAK inhibitor analog is:

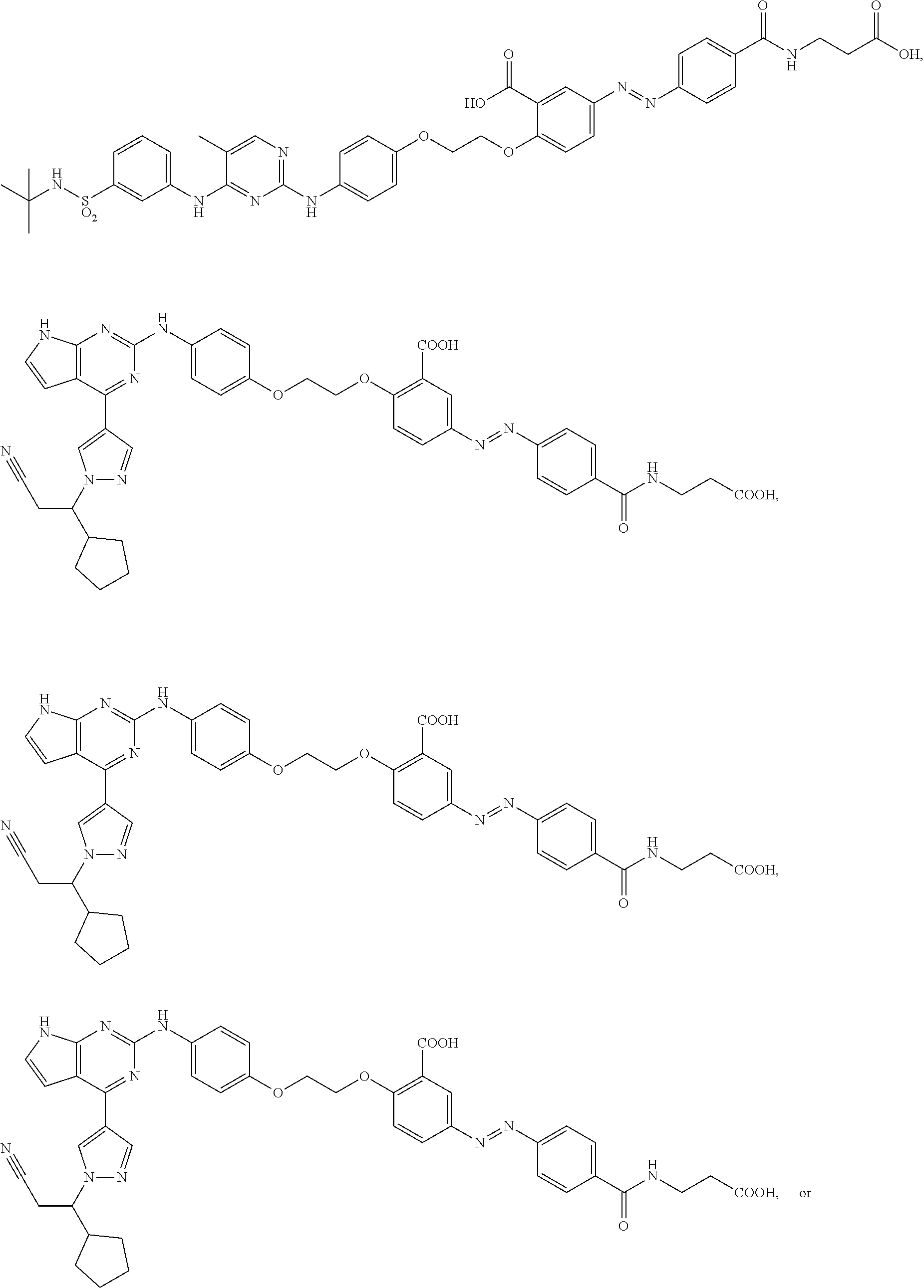 [SMILES: Cc1cnc(Nc2ccc(OCCOc3ccc(/N=N/c4ccc(C(=O)NCCC(=O)O)cc4)cc3C(=O)O)cc2)nc1Nc1cccc(S(=O)(=O)NC(C)(C)C)c1 ; N#CCC(C1CCCC1)n1cc(-c2nc(Nc3ccc(OCCOc4ccc(/N=N/c5ccc(C(=O)NCCC(=O)O)cc5)cc4C(=O)O)cc3)nc3[nH]ccc23)cn1 ; N#CCC(C1CCCC1)n1cc(-c2nc(Nc3ccc(OCCOc4ccc(/N=N/c5ccc(C(=O)NCCC(=O)O)cc5)cc4C(=O)O)cc3)nc3[nH]ccc23)cn1 ; N#CCC(C1CCCC1)n1cc(-c2nc(Nc3ccc(OCCOc4ccc(/N=N/c5ccc(C(=O)NCCC(=O)O)cc5)cc4C(=O)O)cc3)nc3[nH]ccc23)cn1]

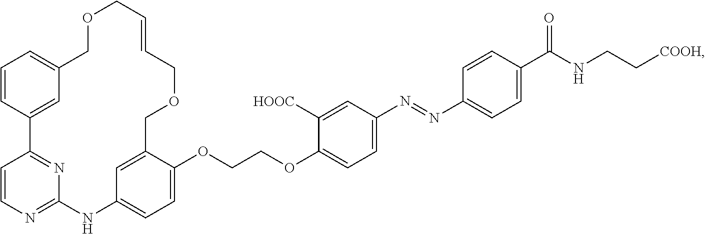 [SMILES: O=C(O)CCNC(=O)c1ccc(/N=N/c2ccc(OCCOc3ccc4cc3COC/C=C/COCc3cccc(c3)-c3ccnc(n3)N4)c(C(=O)O)c2)cc1]

or a pharmaceutically acceptable salt thereof.

In another aspect, disclosed herein is a compound of formula (II):

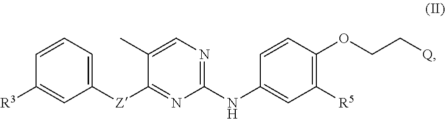

or a pharmaceutically acceptable salt thereof, wherein:

Q is

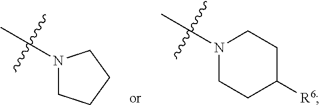

Z′ is NR^{c}, wherein R^{c} is H or C₁-C₆ alkyl;
R³ is alkyl or SO₂—R, wherein R⁴ is selected from C₁-C₆ alkyl, C₃-C₉ cycloalkyl, C₃-C₉ heterocycle, and N(R^{d})₂, and wherein each R^{d} is independently selected from hydrogen, C₁-C₆ alkyl, C₃-C₉ cycloalkyl, and C₃-C₉ heterocycle, or both R^{d} are taken together with the nitrogen atom to which they are attached to form an optionally substituted 5- or 6-membered ring;
R⁵ is hydrogen, —CH₂—OCH₃ or —CH₂—(OCH₂CH₂)—OCH₃; and
R^{A} is —OC-13 or —OCH₂CH₂—OCHI-3.

In some embodiments, Z′ is NH.

In some embodiments, R³ is SO₂—N(R^{d})₂, In some embodiments, each R^{d} is independently C₁-C₆ alkyl. In some embodiments, one R⁴ is hydrogen and one R^{d} is C₁-C₆ alkyl.

In some embodiments, Z′ is NH and R³ is SO₂—N(Rd)₂. In some embodiments, each R^{d} is independently C₁-C₆ alkyl. In some embodiments, one R^{d} is hydrogen and one R^{d} is C₁-C₆ alkyl.

In some embodiments, Q is

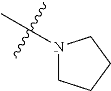 [SMILES: *N1CCCC1]

and R⁵ is —CH₂—OCH₃ or —CH₂—(OCH₂CH₂)—OCH₃.

In some embodiments, Q is

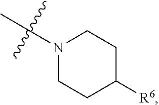

R⁵ is hydrogen, and R⁶ is —OCH₃. In some embodiments, Q is

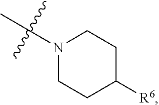

R⁵ is —CH₂—OCH₃, and R⁶ is —OCH₃. In some embodiments, Q is

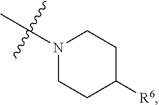

R⁵ is —CH₂—(OCH₂CH₂)—OCH₃, and R⁶ is —OCH₃.

In some embodiments, Q is

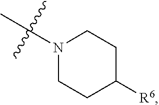

R⁵ is hydrogen, and R⁶ is —OCH₂CH₂—OCH₃. In some embodiments, Q is

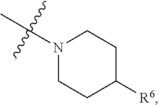

R⁵ is —CH₂—OCH₃, and R⁶ is —OCH₂CH₂—OCH₃. In some embodiments, Q is

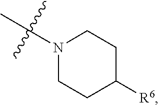

R⁵ is —CH₂—(OCH₂CH₂)—OCH₃, and R⁶ is —OCH₂CH₂—OCH₃.

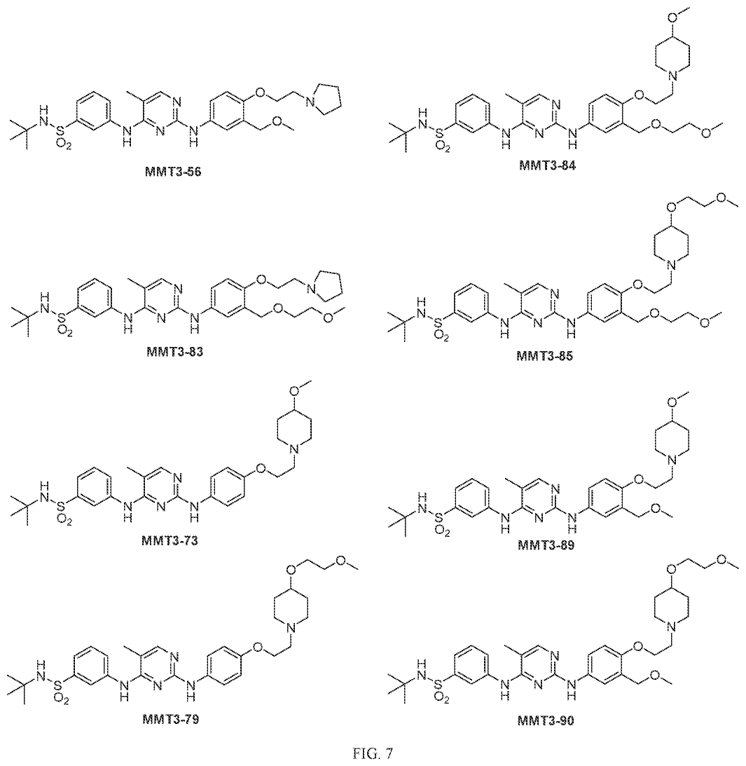

In some embodiments, the compound of formula (II) is a compound shown in FIG. 7, or a pharmaceutically acceptable salt thereof.

The compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England (or more recent versions thereof), or (2) direct separation of the mixture of optical enantiomers on chiral chromatographic columns, or (3) fractional recrystallization methods.

It should be understood that the compounds may possess tautomeric forms, as well as geometric isomers, and that these also constitute embodiments of the disclosure.

The present disclosure also includes isotopically-labeled compounds, which is identical to those recited in formula (I), formula (Ia), or formula (II), but for the fact that one or more atoms are replaced by an atom having an atomic mass or mass number different from the atomic mass or mass number usually found in nature. Examples of isotopes include those for hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, for example, ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in the compounds are ¹¹C, ¹³N, ¹⁵O, and ¹⁸F. Isotopically-labeled compounds can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Compounds may be synthesized according to a variety of methods, including those illustrated in the Examples. Reaction conditions and reaction times for each individual step can vary depending on the particular reactants employed and substituents present in the reactants used Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the disclosure can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the disclosure as it is defined in the appended claims. All alternatives, modifications, and equivalents of the synthetic methods and specific examples are included within the scope of the claims.

### 3. COMPOSITIONS

The disclosed JAK inhibitor analogs may be incorporated into pharmaceutically acceptable compositions. The pharmaceutical compositions may include a “therapeutically effective amount” or a “prophylactically effective amount” of the JAK inhibitor analog(s). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, cyclodextrins combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, tale, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, dimethyl sulfoxide, N-methyl-2-pyrrolidone, dimethylacetamide and phosphate (or other suitable buffer). The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5′%.

Suitable cyclodextrins include alpha-CD, beta-CD, gamma-CD, hydroxypropyl betadex (HP-beta-CD), sulfobutyl-ether β-cyclodextrin (SBE-beta-CD). The amount of cyclodextrins in the systemic or topical composition is typically about 0% to about 40%.

Although the amounts of components in the systemic compositions may vary depending on the type of systemic composition prepared, in general, systemic compositions include 0.01% to 50% of an active compound and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include a compound as disclosed herein, and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT®, coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The composition disclosed herein may further comprise at least one additional therapeutic agent. The at least one additional therapeutic agent may comprise immunosuppressants (e.g., azathioprine, mercaptopurine, cyclosporine, tacrolimus, and methotrexate), anti-inflammatory agents (e.g., corticosteroids and aminosalicylates), chemotherapeutic agents, immunotherapies, antibiotics, anti-diarrheal medications, and analgesics.

### 4. METHODS OF USE

The disclosure further provides methods for treating a disease or disorder comprising administration of a compound (e.g., a JAK inhibitor analog), as disclosed herein, or a composition thereof, to a subject in need thereof. In some embodiments, the JAK inhibitor analog is a compound of formula (I), formula (Ia), or formula (II). In some embodiments, the JAK inhibitor analog is

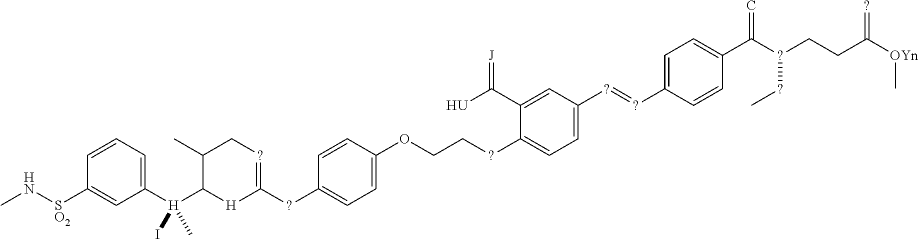

or a pharmaceutically acceptable salt thereof. In some embodiments, the subject is a human.

The disease or disorder may be selected from cancer, autoimmune diseases, and inflammatory diseases.

In some embodiments, the disease or disorder is an inflammatory disease or disorder. Inflammatory diseases are characterized by activation of the immune system in a tissue or an organ to abnormal levels that may lead to abnormal function and/or disease in the tissue or organ. The inflammatory diseases and disorders that may be treated by the methods of the present invention include, but are not limited to, arthritis, rheumatoid arthritis, asthma, inflammatory bowel disease (Crohn's disease or ulcerative colitis), chronic obstructive pulmonary disease (COPD), allergic rhinitis, vasculitis (polyarteritis nodosa, temporal arteritis, Wegener's granulomatosis, Takayasu's arteritis, or Behcet's syndrome), inflammatory neuropathy, psoriasis, systemic lupus erythematosus (SLE), chronic thyroiditis, Hashimoto's thyroiditis, Addison's disease, polymyalgia rheumatica, Sjogren's syndrome, or Churg-Strauss syndrome.

In some embodiments, the disease or disorder is an autoimmune disease or disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS) chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis, Raynaud's phenomenon, Reiter's syndrome. Rheumatoid arthritis, sarcoidosis, scleroderma, Sjogren's syndrome, stiff-man syndrome, systemic lupus erythematosus, lupus erythematosus, Takayasu arteritis, temporal arteritis/giant cell arteritis, ulcerative colitis, uveitis, vasculitides such as dermatitis herpetiformis vasculitis, vitiligo, and Wegener's granulomatosis.

Some autoimmune disorders are also associated with an inflammatory condition. Examples of inflammatory disorders which are also autoimmune disorders that can be prevented, treated or managed in accordance with the methods of the invention include, but are not limited to, asthma, encephalitis, inflammatory bowel disease, chronic obstructive pulmonary disease (COPD), allergic disorders, pulmonary fibrosis, undifferentiated spondyloarthropathy, undifferentiated arthropathy, arthritis, inflammatory osteolysis, and chronic inflammation resulting from chronic viral or bacterial infections. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis.

In some embodiments, the disease or disorder is a gastrointestinal inflammatory disease or disorder. These diseases or disorders include, for example, inflammatory bowel disease (e.g., Crohn's disease, ulcerative colitis, indeterminate colitis, and infectious colitis), mucositis (e.g., oral mucositis, gastrointestinal mucositis, nasal mucositis and proctitis), necrotizing enterocolitis and esophagitis. Generally, gastrointestinal inflammatory diseases or disorders include any disease or disorder that comprises cause inflammation and/or ulceration in the mucous membrane of the gastrointestinal tract.

In some embodiments, the disease or disorder is cancer. In some embodiments, the cancer comprises a solid tumor. In some embodiments, the cancer comprises a blood cancer or lymphoma. In some embodiments, the cancer is metastatic cancer. In some embodiments, the disclosed compounds, compositions, or methods result in suppression of elimination of metastasis. In some embodiments, the disclosed compounds, compositions, or methods result in decreased tumor growth. In some embodiments, the disclosed compounds, compositions, or methods prevent tumor recurrence.

The compounds and compositions herein may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus.

In some embodiments, the cancer is invasive and/or metastatic cancer (e.g., stage IT cancer, stage III cancer or stage IV cancer). In some embodiments, the cancer is an early stage cancer (e.g., stage 0 cancer, stage I cancer), and/or is not invasive and/or metastatic cancer.

The JAK inhibitor analog, or a composition thereof, may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. In some embodiments, the JAK inhibitor analog, or a composition thereof, as disclosed herein may be administered by oral administration.

The amount of the JAK inhibitor analog, or a composition thereof, of the present disclosure required for use in treatment or prevention will vary not only with the particular compound selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a JAK inhibitor analog, or a composition thereof, can be determined by comparing their in vitro activity, and in vivo activity in animal models.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, w ill also vary according to the age, body weight, and response of the individual patient. A program comparable to that discussed above may be used in veterinary medicine.

JAK inhibitor analogs, or compositions thereof, disclosed herein can be evaluated for efficacy and toxicity using known methods. For example, the toxicology of a particular compound, a subset of the compounds sharing certain chemical moieties, or a composition comprising a JAK inhibitor analog, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs, or monkeys, may be determined using known methods. Efficacy may be established using several recognized methods, such as in vitro methods, animal models, or human clinical trials. When selecting a model to determine efficacy, the skilled artisan can be guided by the state of the art to choose an appropriate model, dose, route of administration and/or regime.

A wide range of second therapies may be used in conjunction with the compounds and methods of the present disclosure. The second therapy may be administration of an additional active agent or may be a second therapy not connected to administration of another agent. Such second therapies include, but are not limited to, surgery, immunotherapy, radiotherapy.

The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months.

A therapeutically effective amount of a JAK inhibitor analog or compound disclosed herein, or compositions thereof, may be administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time, wherein one composition includes a compound of this invention, and the other includes the second agent(s).

The at least one additional therapeutic agent may comprise immunosuppressants (e.g., azathioprine, mercaptopurine, cyclosporine, tacrolimus, and methotrexate), anti-inflammatory agents (e.g., corticosteroids and aminosalicylates), chemotherapeutic agents, immunotherapies, antibiotics, anti-diarrheal medications, and analgesics.

In some embodiments, the at least one additional therapeutic agent comprises at least one chemotherapeutic agent. As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemaciclib, everolimus, alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib. In select embodiments, the chemotherapeutic agent comprises paclitaxel.

In some embodiments, the second therapy includes immunotherapy. Immunotherapies include chimeric antigen receptor (CAR) T-cell or T-cell transfer therapies, cytokine therapy, immunomodulators, cancer vaccines, or administration of antibodies (e.g. monoclonal antibodies).

In some embodiments, the immunotherapy comprises administration of antibodies. The antibodies may target antigens either specifically expressed by tumor cells or antigens shared with normal cells. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like), In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The immunotherapy (e.g., administration of antibodies) may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. In some embodiments, the immunotherapy may be administered in the same or different manner than the JAK inhibitor analog, or composition thereof. The immunotherapy may be administered by parenteral administration (including, but not limited to, subcutaneous, intramuscular, intravenous, intraperitoneal, intracardiac and intraarticular injections).

### 5. KITS

In another aspect, the disclosure provides kits comprising at least one disclosed JAK inhibitor analog or a pharmaceutically acceptable salt thereof, or a composition comprising the compound or a pharmaceutically acceptable salt thereof, and instructions for using the compound or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed compound and/or product and another agent (e.g., a chemotherapeutic, a monoclonal antibody, a pain reliever, an immunosuppressants, an anti-inflammatory agent, an antibiotic, an anti-diarrheal) for delivery to a patient.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

The kits provided herein are in suitable packaging. Suitable packaging includes, but is not limited to, vials, bottles, jars, flexible packaging, and the like. Individual member components of the kits may be physically packaged together or separately.

### 6. EXAMPLES

Abbreviations used in the schemes and examples that follow are: DCE is dichloroethane; DMA is dimethylacetamide; DMF is dimethylformamide; DMSO is dimethyl sulfoxide: EtOAc is ethyl acetate; EtOH is ethanol: HCl is hydrochloric acid; iPrOH is isopropylalcohol; MeOH is methanol: Pd/C is palladium on carbon; and rt is room temperature.

All commercially available products and solvents were purchased from Sigma-Aldrich, AK Scientific, and Fisher scientific. Solvents were used as received or dried over molecular sieves (4 Å). All water or air-sensitive reactions were performed under an argon atmosphere with dry solvents and anhydrous conditions. All reactions were monitored by thin-laver chromatography (TLC) that were performed on aluminum-backed silica plates (0.2 mm, 60 F254). Purification by flash chromatography was performed on Merck silica gel 60 (230-400 mesh). Yields refer to chromatographically and spectroscopically (1H NMR) homogeneous materials, unless otherwise stated.

NMR spectra were recorded on a Bruker instrument (500 or 300 MHz) and calibrated using a solvent peak as an internal reference. Spectra were processed using MestReNova software. Chemical shifts δ are given in ppm and coupling constants (J) in Hz. Peak multiplicities are described as follows: s, singlet, t, triplet, and m, multiplet. High-resolution mass spectra were obtained on an AB Sciex X500R QTOF spectrometer or an AB Sciex 6600+ Triple TOF mass spectrometer. The purity of all compounds subjected to biological tests was determined by analytical HPLC and was found to be ≥95%.

### Example 1

### Compound Synthesis

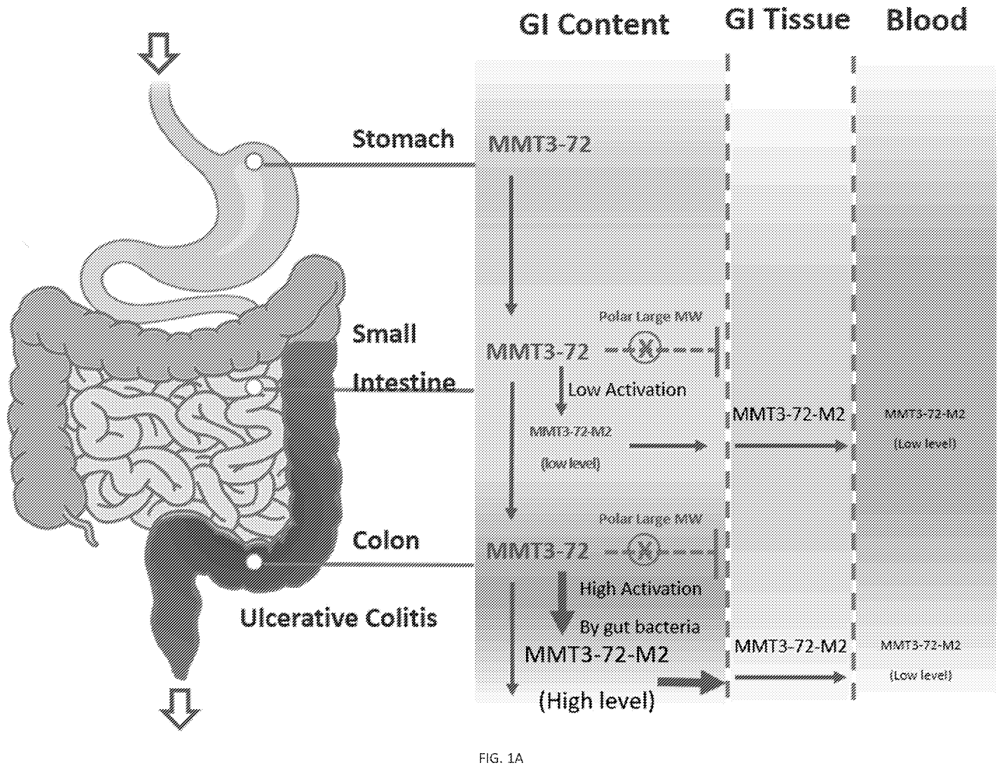

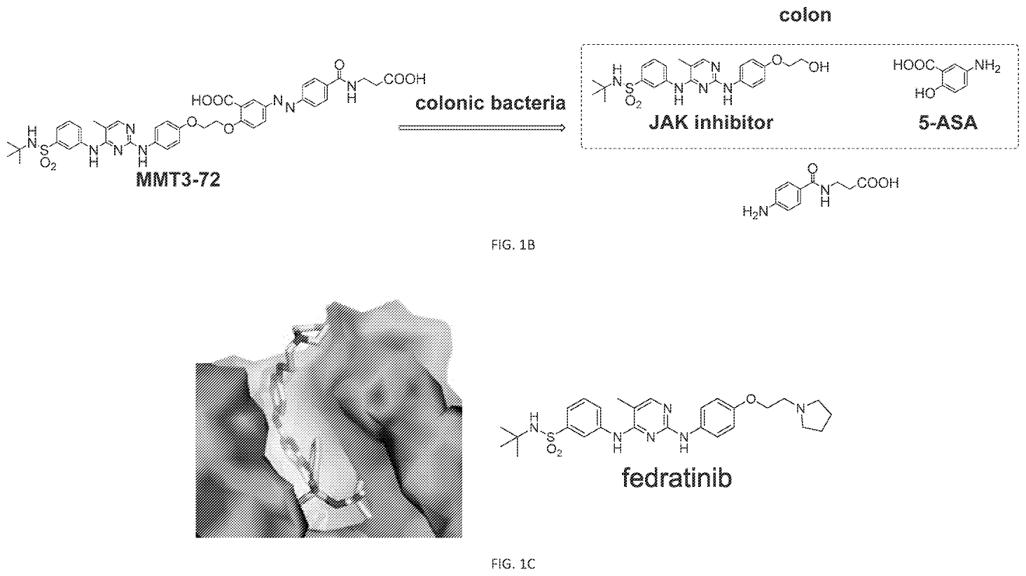

The compound MMT3-72 was designed using fedratinib scaffold (FIG. 1A). Fedratinib is a semi-selective JAK2 inhibitor. Fedratinib inhibits JAK2 (IC₅₀ 15 nM) while it also inhibits JAK1 (IC₅₀ 10 nM) and TYK2 (IC₅₀ 178 nM). JAK2 and TYK2 are the JAK isoforms involved in the IL-12/IL-23 signaling, and inhibition of IL-12/IL-23 (such as antibody Ustekinumab) is effective in the treatment of UC. A large portion (77%) of fedratinib is excreted to GI tract in feces (23% as unchanged drug) following a single oral dose.

The solvent exposed pyrrolidine moiety of fedratinib was replaced in MMT3-72 with 5-aminosalicylic acid (5-ASA) linked by an azo bond to N-4-(aminobenzoyl)-beta-alanine. The azo bond can be cleaved by colonic bacteria to release the active metabolite in the GI tract that is absorbed and accumulated in the colon tissues with minimal exposure in the systemic circulation.

The compounds MMT3-72 and MMT3-72-M2 were synthesized according to the synthetic route as shown in Scheme 1. Briefly, 4-nitrophenol was condensed with DCE or 2-chloride ethanol to produce intermediates 1 and 4, respectively. A subsequent nitro reduction of intermediates 1 and 4 with tin chloride or from hydrogenation afforded the intermediates 2 and 5, respectively. Then, intermediate 2 or 5 was coupled with N-tert-butyl-3-[(2-chloro-5-methylpyrimidin-4-yl)amino]benzenesulfonamide in the presence of a few drop of HCl in isopropanol to generate compound 3 and MMT3-72-M2, respectively, Finally, compound 3 was subject to a nucleophilic substitution reaction with balsalazide disodium salt dihydrate to yield the desired compound MMT3-72.

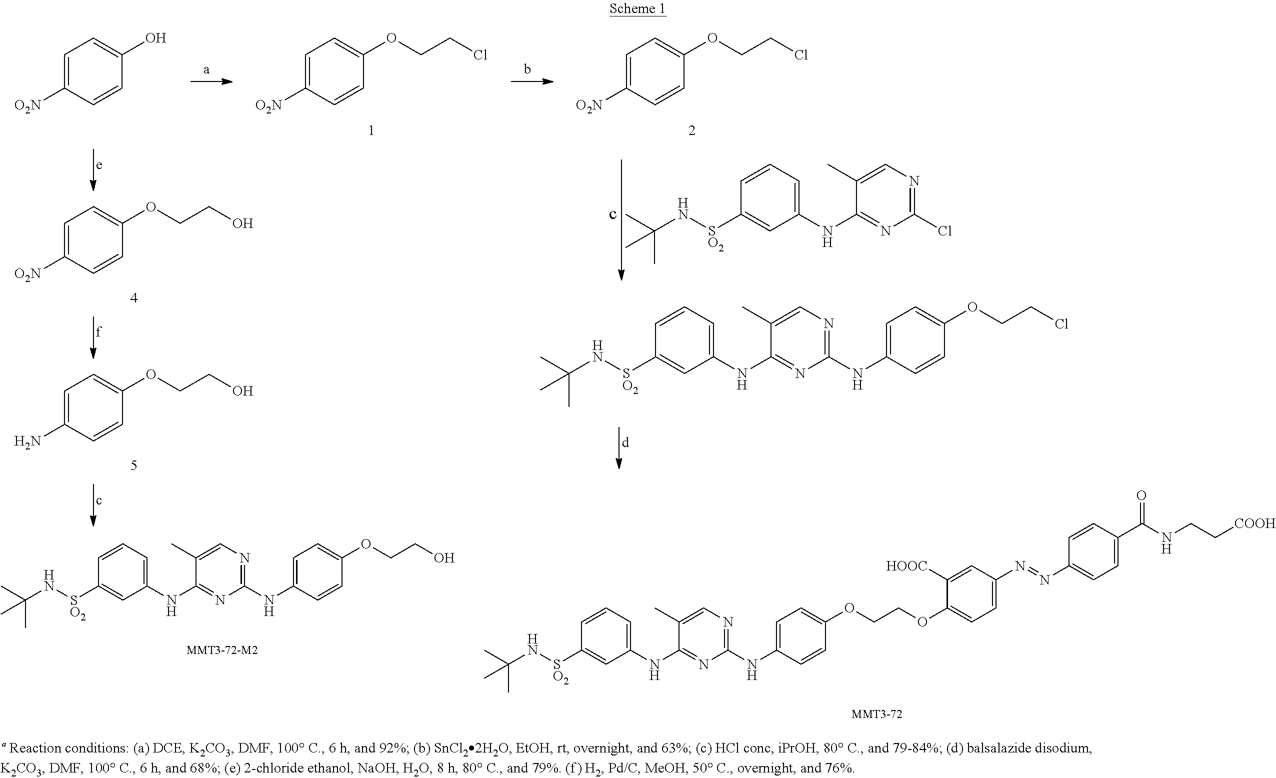

N-(tert-butyl)-3-((2-((4-(2-hydroxyethoxy)phenyl)amino)-5-methylpyrimidin-4-yl)amino)benzenesulfonamide (MMT3-72-M2): To a mixture of compound 5 (130 mg, 0.846 mmol, 3 equiv) and N-tert-butyl-3-[(2-chloro-5-methylpyrimidin-4-yl)amino]benzenesulfonamide (100 mg. 0.282 mmol) in isopropanol (2 mL) was added 3 drops of concentrated HCl 37% and the reaction mixture was stirred at 80° C. for overnight. Upon completion of the reaction, the solvent was evaporated under reduced pressure and the residue was taken into aqueous NaHCO₃ and extracted three times with CH₂Cl₂. The combined organic phase was washed with water, brine and dried over Na₂SO₄. The solvent was concentrated tinder vacuum and the obtained solid was washed three times with EtOAc to provide the compound MMT3-72-M2 (105 mg, 79% yield). ¹H NMR (300 MHz, DMSO-d₆) δ 8.79 (s, 1H), 8.55 (s, 1H), 8.12 (d, J:=3.4 Hz, 2H), 7.90 (s, 1H), 7.62-7.39 (m, 4H), 6.80 (d, J=8.9 Hz, 2H), 3.92 (t, J=5.1 Hz, 2H), 3.69 (t, J=5.0 Hz, 2H), 2.12 (s, 3H), 1.12 (s, 9H). HRMS (ESI): mass calcd. for CN₂₃H₂₉N₅O₄S, 471.58; m/z found, 472.1847 [M+H]+.

The synthesis and characterization of compounds 1 and 4 have been previously described (See, Luo, et al., Bioorg Med Chem Lett 2017, 27 (12), 2668-2673).

1-(2-chloroethoxy)-4-nitrobenzene (1): To a mixture of 4-nitrophenol (4 g, 28.754 mmol) and 1,2-dichloromethane (20 mL, 5 vol) in DMF (25 ml) was added K₂CO₃ (6 g, 43.131 mmol, 1.5 equiv), and the resulting mixture was stirred at 100° C. for 6 h and monitored by TLC. Upon completion, the reaction mixture was quenched with water and the product was extracted three times with CH₂Cl₂. The combined organic phase was washed with water, brine, dried over Na₂SO₄, and concentrated under vacuum to give compound 1 (5.35 g; yield, 92%). This intermediate was taken forward to the next step without further purification.

4-(2-chloroethoxy)aniline (2): To a mixture of compound 1 (1 g, 4.96 mmol) in EtOH (30 mL) was added SnCl₂·2H₂O (4.5 g, 19.84 mmol, 4 equiv) and the reaction mixture was stirred at 90° C. for overnight. Upon completion of the reaction, the solvent was evaporated under reduced pressure and the residue was taken into 5% aqueous NaOH and extracted three times with CH₂Cl₂. The combined organic phase was washed with 5% aqueous NaOH, water, brine and dried over Na₂SO₄. The solvent was concentrated under vacuum and the residue was purified by silica gel column chromatography to provide compound 2 (532.4 mg, 63% yield).

¹H NMR (500 MHz, CDCl₃) δ 6.82-6.69 (m, 2H), 6.69-6.58 (m, 2H), 4.16 (t, J=5.9 Hz, 2H), 3.77 (t, J:=5.9 Hz, 2H). HRMS (ESI): mass calcd. for C₈H₁₀ClNO. 171.05; m/z found. 172.0425 [M+H]+.

N-(tert-butyl)-3-((2-((4-(2-chloroethoxy)phenyl)amino)-5-methylpyrimidin-4-yl)amino)benzenesulfonamide (3): To a mixture of compound 2 (400 mg, 2.330 mmol, 2 equiv) and N-tert-butyl-3-[(2-chloro-5-methylpyrimidin-4-yl)amino]benzenesulfonamide (413.53 mg, 1.165 mmol) in isopropanol (8 mL) was added 3 drops of concentrated HCl 37% and the reaction mixture was stirred at 80° C. for overnight. Upon completion of the reaction, the solvent was evaporated under reduced pressure and the residue was taken into aqueous NaHCO₃ and extracted three times with CH₂C₂. The combined organic phase was washed with water, brine and dried over Na₂SO₄. The solvent was concentrated under vacuum and the obtained solid was washed three times with EtOAc to provide the compound 3 (482 mg, 84% yield).

¹H NMR (500 MHz, DMSO-d₆) δ 8.82 (s, 1H), 8.55 (s, 1H), 8.12 (d, J=5.6 Hz, 2H), 7.91 (d, J=1.0 Hz, 1H), 7.56 (d, J=4.3 Hz, 2H), 7.51-7.45 (m, 2H), 6.87-6.80 (m, 2H), 4.18 (d, J=6.0 Hz, 2H), 3.92 (d, J=5.9 Hz, 2H), 216-2.06 (m, 3H), 1.12 (s, 9H). HRMS (EST): mass calcd. for C₂₃H₂₈ClN₅O₃S, 489.16; m/z found, 490.1506 [M+H]+.

2-(4-nitrophenoxy)ethan-1-ol (4): To a mixture of 4-nitrophenol (3 g, 21.56 mmol) and 2-chloride ethanol (2.89 mL, 4316 mmol, 2 equiv) in H₂O (10 mL) was added NaOH (1.73 g, 43.16 mmol, 2 equiv) and the reaction mixture was stirred at 80° C. for overnight. Upon completion of the reaction, the reaction mixture was cooled down to room temperature, diluted with H₂O and extracted three times with EtOAc. The combined organic phase was washed with water, brine and dried over Na₂SO₄. The solvent was concentrated under vacuum to give compound 4 (3.1 g, 79% yield), which was taken forward to the next step without further purification.

2-(4-aminophenoxy)ethan-1-ol (5): To a mixture of compound 4 (1 g, 5.460 mmol) in MeOH (20 mL) was added Pd/C (0.1 g, 10% equiv) and the reaction mixture was stirred at 50° C. for overnight under H₂ atmosphere. Upon completion of the reaction, the Pd/C was filtered off on celite and the solvent was evaporated under reduced pressure. The residue was purified by silica gel column chromatography to provide compound 5 (635.2 mg, 76% yield).

¹H NMR (300 MHz, CDCl₃) δ 6.82-6.72 (m, 2H), 6.70-6.59 (m, 2H), 4.01 (dd, J=5.1, 3.5 Hz, 2H), 3.92 (dd, J=5.1, 3.5 Hz, 2H). HRMS (ESI): mass calcd. for C₈H₁₀NO₂, 153.18; m/z found, 154.0770 [M+H]+.

(E)-2-(2-(4-((4-((3-(N-(tert-butyl)sulfamoyl)phenyl)amino)-5-methylpyrimidin-2-yl)amino)phenoxy)ethoxy)-5-((4-((2-carboxyethyl)carbamoyl)phenyl)diazenyl)benzoic acid (MMT3-72): To a mixture of compound 3 (45 mg, 0.09 mmol, 1.5 equiv) and balsalazide disodium salt dehydrate (27.29 mg, 0.068 mmol, 1 equiv) in DMF (2 mL) was added K₂CO₃ (37.6 mg, 0.272 mmol, 4 equiv), and the resulting mixture was stirred at 100° C. for overnight and monitored by TLC. Upon completion, the solvent was evaporated under reduced pressure. The residue was taken into 1120 and the solution was acidified with H₃PO₄ until pH 2-3. The precipitate was filtered and recrystallization in CH₂Cl₂ provided the desired compound MMT3-72 (50.4 mg, 68%).

¹H NMR (500 MHz, DMSO-d₆) δ 8.68 (s, 1H), 8.29 (s, 1H), 8.08 (s, 2H), 7.96 (d, J=8.3 Hz, 2H), 7.88 (d, J=10.5 Hz, 2H), 7.82 (d, J=8.3 Hz, 2H), 7.57 (s, 1H), 7.49 (t, J:=14.4 Hz, 4H), 6.86 (d, J=9.7 Hz, 1H), 6.80 (d, J=8.5 Hz, 2H), 4.36 (t, J=4.5 Hz, 2H), 4.13 (t, J=4.8 Hz, 2H), 3.61-3.48 (m, 2H), 2.66 (t, J=6.9 Hz, 2H), 2.11 (s, 3H), 1.11 (s, 9H), HRMS (ESI): mass calcd. for C₄₀H₄₂N₈O₉S, 810.2; m/z found, 811.2758 [M+H]+.

The compounds MMT3-83, MMT3-84, and MMT3-85 were synthesized according to the synthetic route as shown in Scheme 2. Compound MMT3-56 was synthesized by methods similar to those shown for MMT3-83. Compounds MMT13-73 and MMT3-79 were synthesized according to methods similar to those shown for MMT3-72. Compounds MMT3-89 and MMT3-90 were synthesized according to methods similar to those shown for MMT3-84 and MMT3-85.

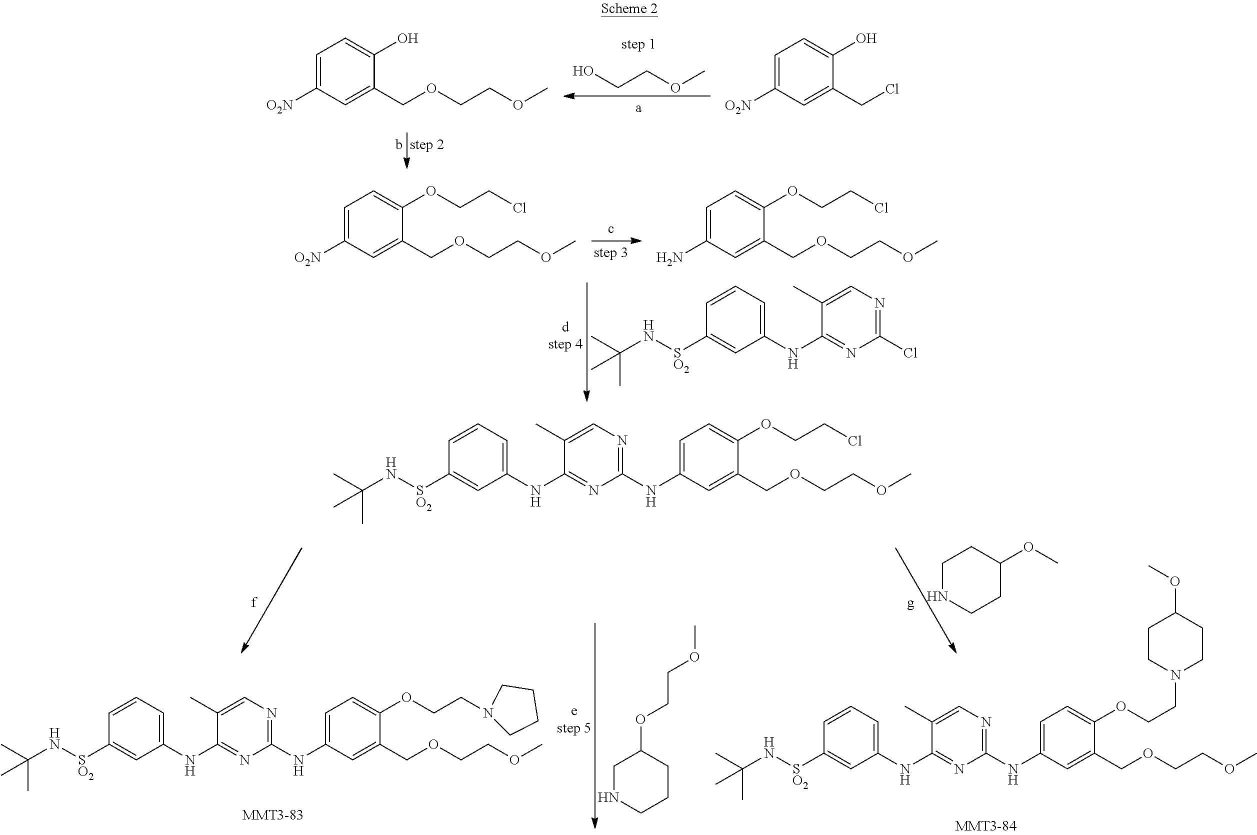

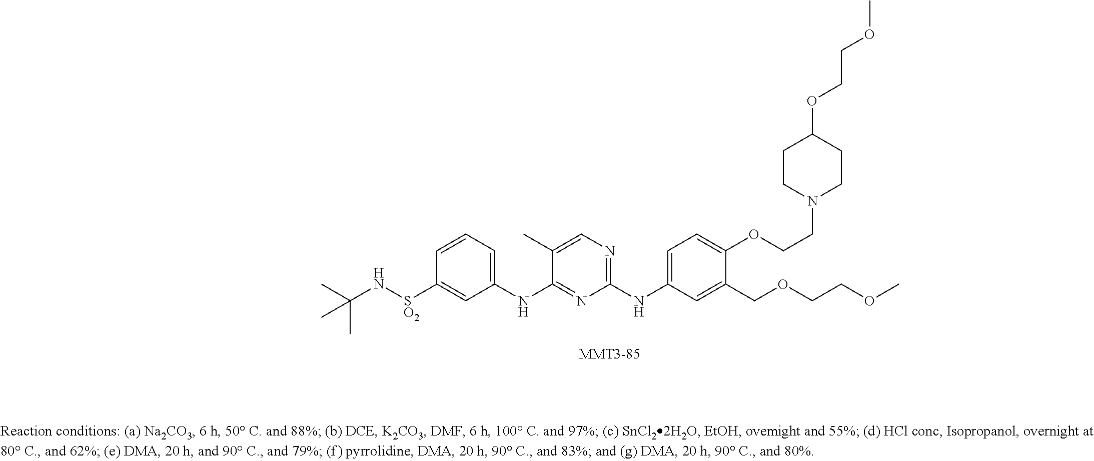 [SMILES: COCCOCc1cc(Nc2ncc(C)c(Nc3cccc(S(=O)(=O)NC(C)(C)C)c3)n2)ccc1OCCN1CCC(OCCOC)CC1]

Step 1. A solution of 2-(chloromethyl)-4-nitrophenol (0.25 g; 1.33 mmol) in 5 mL of 2-methoxyethanol or appropriate alcohol was heated to 50° C. and stirred under argon. Sodium bicarbonate (0.23 g; 2.66 mmol, 2 equiv) was added gradually over 1 h and the reaction was allowed to proceed for 7 h at 50° C. Upon completion of the reaction, the redundant NaHCO₃ was removed by filtration, the alcohol was evaporated, and the product was crystallized from ethyl acetate (yields 88-97%).

Step 2. To a mixture of intermediate obtained from step 1 (1.95 g; 7.03 mmol) and 1,2-dichloromethane (10 mL, 5 vol) in DMF (25 mL) was added K₂CO₃ (1.46 g, 10.55 mmol, 1.5 equiv), and the resulting mixture was stirred at 100° C. for 6 h and monitored by TLC. Upon completion, the reaction mixture was quenched with water and the product was extracted three times with CH₂Cl₂. The combined organic phase was washed with water, brine, dried over Na₂SO₄, and concentrated under vacuum to give the product (1.97 g; yield, 97%). This intermediate was taken forward to the next step without further purification.

Step 3. To a mixture of intermediate obtained from step 2 (1 g, 3.45 mmol) in EtOH (30 mL) was added SnCl₂·2H₂O (3.11 g, 13.807 mmol, 4 equiv) and the reaction mixture was stirred at 90° C. for overnight. Upon completion of the reaction, the solvent was evaporated under reduced pressure and the residue was taken into 5% aqueous NaOH and extracted three times with CH₂Cl₂. The combined organic phase was washed with 5% aqueous NaOH, water, brine and dried over Na₂SO₄. The solvent was concentrated under vacuum and the residue was purified by silica gel column chromatography to provide the product (0.5 g, 55% yield).

Step 4. To a mixture of intermediate obtained from step 3 (450 mg, 1.73 mmol, 2 equiv) and N-tert-butyl-3-[(2-chloro-5-methylpyrimidin-4-yl)amino]benzenesulfonamide (307 mg, 0.86 mmol) in isopropanol (8 mL) was added 3 drops of concentrated HCl 37% and the reaction mixture was stirred at 80° C. for overnight. Upon completion of the reaction, the solvent was evaporated under reduced pressure and the residue was taken into aqueous NaHCO₃ and extracted three times with CH₂Cl₂. The combined organic phase was washed with water, brine and dried over Na₂SO₄, The solvent was concentrated under vacuum and the obtained solid was washed three times with EtOAc to provide the product (311.7 mg, 62% yield).

Step 5. To a mixture of intermediate obtained from step 4 (80 mg. 0.138 mmol) in DMA (2 mL) was added appropriate amine (2 vol) and the reaction mixture was stirred at 90° C. for overnight. Upon completion of the reaction, the solvent was evaporated under reduced pressure and the residue was purified by silica gel column chromatography to provide the product (yields 80-95%).

### Example 2

### GI Local Activation of MMT3-72 and Metabolites Identification in GI Content, CI Tissues and Plasma

### Materials and Methods

Activation of MMT73-72 and Metabolite identification: In-vivo metabolite identification was conducted using mouse plasma, colon and feces samples that collected at 6 h after oral administration of MMT3-72 (10 mg/kg), Liquid chromatography tandem mass spectrometry was employed to separate and identify the possible metabolites. The LC-MS/MS method consisted of a Shimadzu LC-20AD HPLC system (Kyoto, Japan). Chromatographic separation of MMT3-72 and its metabolites was achieved using a Waters XBridage reverse phase C18 column (15 cm×2.1 mm I.D., packed with 3.5 m). A high resolution AB Sciex X500R QTOF mass spectrometer (AB Sciex, Farmingham, USA) in the positive-ion Information Dependent Acquisition (IDA) mode was used for confirmation of accurate molecular weight. The mass range was recorded from m/z 100 to 1000 Da. The collision energy was set to 50 V for TOF MSMS. Data was collected with software SCIEX OS, and then processed with software Metabolite Pilot 2.0 (AB Sciex, Farmingham, USA).

In vitro activity to inhibit JAK enzymes: JAK1, JAK2, JAK3, TYK2 assay kits were obtained from BPS Bioscience (San Diego, CA, USA). The assays were conducted according to manufacturer's protocols in 96-well microplates. Briefly, master mixtures (25 μL per well) were prepared for JAK1 and TYK2 assays (6 μL 5× kinase assay buffer+1 μL ATP (500 μM)+5 μL×10 IRS1-tide+13 μL distilled water) or for JAK2 and JAK3 assays (6 μl 5× kinase assay buffer+1 μl ATP (500 μM)+1 μl PTK substrate Poly(Glu:Tyr 4:1) (10 mg/mL)+17 μL distilled water), respectively. Then 5 μL of Fedratinib, MMT3-73-72, MMT3-72-M2 solutions at different concentrations were added to the above prepared master mixtures, which were followed by 20 μL of enzymes (JAK1 at 5 ng/μL, JAK2 at 2.5 ng/μL, JAK3 at 0.4 ng/μL or TYK2 at 0.5 ng/μL), respectively. The reaction mixtures were incubated at 30° C. for 40 minutes. Finally, 50 μL of Kinase-Glo Max reagent (Promega, Madison, WL USA) was added to each well and the reactions were performed in darkness for 15 minutes at room temperature. The luminescence of the reaction mixture was read on a Synergy 2 microplate reader (Biotek).

LC-MS analysis of MMT3-72and MMT3-72-M2 in biological samples: MMT3-72 and MMT3-72-M2 concentrations in plasma (ng/mil) and tissues (ng/g) were determined by the LC-MS/MS method that was developed and validated for this study. The HPLC method was conducted on a Shimadzu LC-20AD HPLC system (Kyoto, Japan), and chromatographic separation was achieved using a Waters XBridage reverse phase C18 column (5 cm×2.1 mm I.D., packed with 3.5 μm). The flow rate of gradient elution was 0.4 ml/min with mobile phase A (0.1% formic acid in purified deionized water) and mobile phase B (0.1% formic acid in acetonitrile). An AB Sciex QTrap 4500 mass spectrometer (AB Sciex, Framingham, USA) in the positive-ion multiple reaction monitoring (MRM) mode was used for detection. Protonated molecular ions and the respective ion products were monitored at the transitions of m/z 811.3>737.4 for MMT3-72 and 472.3>416.0 for the MMT3-72-M2. Data was processed with software Analyst (version 1.6).

Pharmacokinetics in mice. Briefly, C57BL/6 female mice were orally dosed with 10 mg/kg MMT3-72. At 0.5, 2, 4, 12, 24 h, mice were sacrificed, and blood samples were collected by drawing directly from the heart Intestinal tissue samples were collected and homogenized in PBS to 10% homogenate. The contents of small and large intestine were collected and homogenized in PBS. Afterwards, concentrations of MMT3-72, MMT3-72-M2,5-ASA in the plasma, intestinal tissues, and intestinal content were determined by liquid chromatography-tandem mass spectrometry (LC-MS/MS) analysis using method described above.

In vivo efficacy of MMT3-72 in treatment of DSS-induced colitis in mice: C57BL/6 female mice 6-8 weeks purchased from Charles River Laboratories and were randomly divided into different treatment groups. Acute colitis was induced by administering 3% DSS (MP Biomedicals, CA, USA) in distilled water continuously for 5 days and the control group received pure water (Snider, et al., Methods Mol Biol 2016, 1438, 245-254). MMT3-72 or Tofacitinib were dissolved in beta-cyclodextrin. Drugs were administered every other day orally by gavage in volume of 0.1 mL/10 g body weight. During the model establishment, body weight, stool consistency and gross blood in feces were monitored and recorded daily. After 5 days, mice were sacrificed and blood was collected. Serum was obtained by centrifugation and stored at −80° C. for further immune assay. Colon was excised and the length was measured.

H&E Staining of Colon tissues: After dissecting and transecting the colon, a feeding needle and 5 ml syringe were used to intubate and flush the colon with ice-cold PBS until the stool was flushed out. Scissors were used to incise longitudinally from distal to proximal end of the colon and the colon tissue could then be expanded as a flat sheet. The edge of the distal colon was grasped using a pair of forceps and the colon tissue was rotated into a swiss roll. The roll was firmly grasped and transected using a 27 G ½ needle. Then the sample was placed in 4% Paraformaldehyde Fix Solution (Thermo Scientific, USA) at room temperature for 24 hours. The Swiss roll was then paraffin-embedded, sectioned, mounted, and stained with H&E to determine the extent of damage to the colon from the distal (inside end) to the proximal (outside end).

### GI Local Activation of MMT3-72 and Metabolites Identification in GI Content, GI Tissues and Plasma

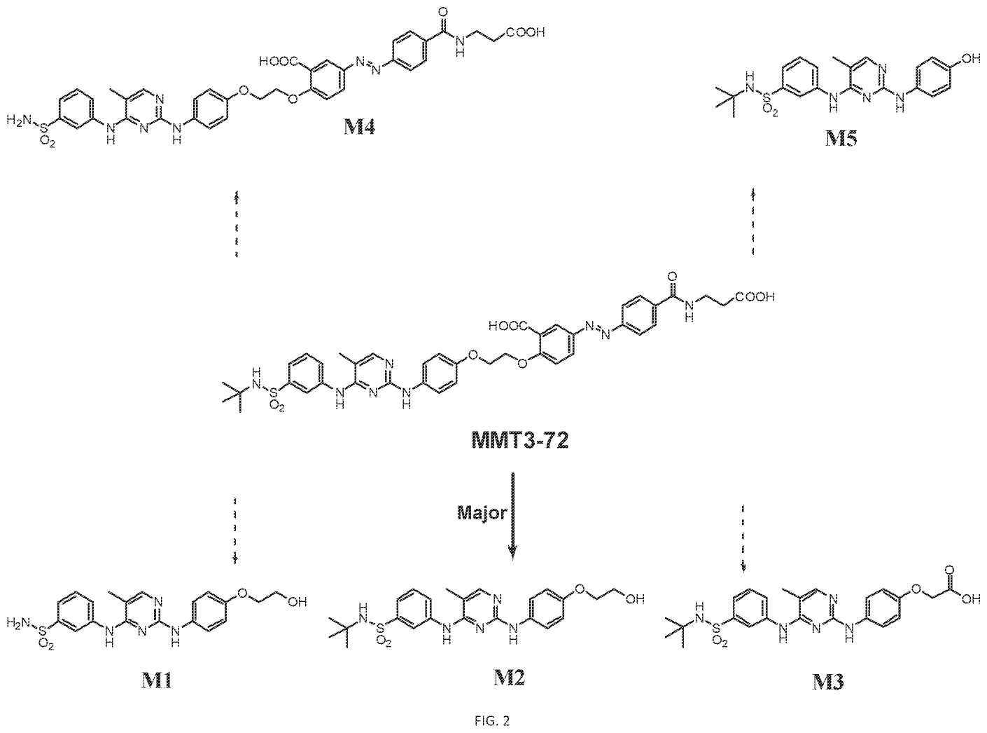

To confirm the activation of MMT3-72 in the colon, a mouse was dosed orally with 10 mg/kg MMT3-72 and sacrificed at 6 h to collect plasma, colon tissue, and colon content (feces). Five metabolites (M1 to M5) were identified in the collected samples and their structure is shown in FIG. 2. Interestingly, MMT3-72 was only detected in the feces with no detection in the plasma and the colon tissues. The major metabolite MMT3-72-M2 was only detected in the colon tissues and the feces with low level in the plasma. Other four minor metabolites M1, M3, M4 and M5 were only identified in the feces with no detection in the plasma and the colon tissues. Since MM T3-72-M2 is the major metabolite and is accumulated in the colon tissue, MMT3-72-M2 was synthesized following the synthetic route shown in Scheme 1 to test its activity in inhibiting JAK1-3 and TYK2 (Table 1).

### MMT3-72 is Less Active but MMT3-72-M2 is More Potent Against JAK1, 2, and TYK2 by In Vitro Kinase Assays

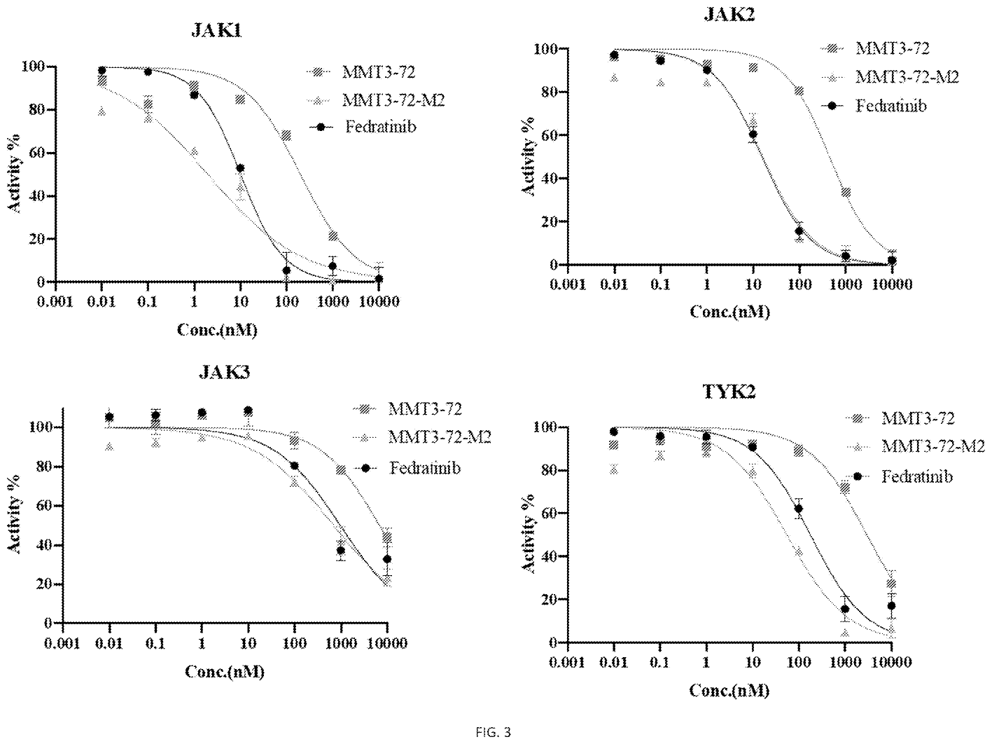

Biological activities of the MMT3-72 and its active metabolite MMT3-72-M2 were evaluated against JAK1, JAK2, JAK3 and TYK2 using kinase assays (FIG. 3, Table 1). The compound MMT3-72 showed modest inhibitory activities against JAK1 and JAK2 (199.3 nM and 448.3 nM, respectively) and poor inhibitory activities against JAK3 and TYK2 (6821 nM and 2976 nM, respectively). However, the active metabolite MMT3-72-M2 showed strong inhibitory activities against JAK1 (2.0 nM), JAK2 (16.3 nM), and TYK2 (55.2 nM), but only weak inhibitory activities against JAK3 (701.3 nM). In comparison, fedratinib strongly inhibited JAK1 (10.1 nM) and JAK2 (15.6 nM), but poorly inhibited JAK3 and TYK2. The inhibitory profiles of JAK1, 2, and TYK2 of MMT3-72-M2 may have advantages to treat UC since JAK2/TYK2/IL-12/IL-23 signaling is strongly implicated in UC, while JAK1 isoform has long been identified as potential target in treating IBD as seen in Upadacitinib. In addition, MMT3-72-M2 showed poor inhibitory activities against JAK3 that may also be preferred in treating UC to reduce the unwanted adverse effects. Tofacitinib inhibited JAK3 with an IC50 of 1.6 nM and showed serious adverse effects. JAK3 inhibition has been shown to potentially lead to lymphopenia and thus hypothetically to an increased risk of infection.

TABLE 1
In vitro inhibitory activities (IC50) of MMT3-72 and active
metabolite MMT3-72-M2 against different isoforms of JAKs

Enzyme IC₅₀ (nM)

| Compounds | JAK1 | JAK2 | JAK3 | TYK2 |
|---|---|---|---|---|
| MMT3-72 | 199.3 | 448.3 | 6821 | 2976 |
| MMT3-72-M2 | 2.0 | 16.3 | 701.3 | 55.2 |
| Fedratinib | 10.1 | 15.6 | 976.5 | 178.5 |
| Tofacitinib | 3.2 | 4.1 | 1.6 | 34.0 |

MMT3-72 was Locally Activated in the CI Tract to Release Active Metabolite MMT3-72-M2 that Achieved High Exposure in the GI Tissues and Minimized Exposure in the Plasma.

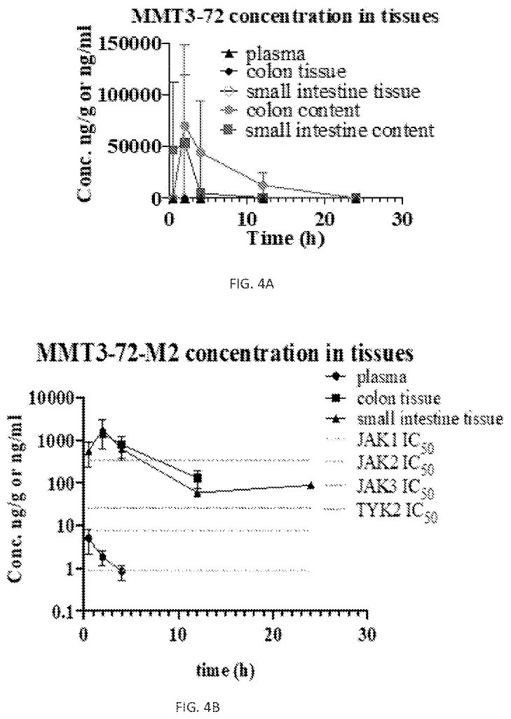

To investigate the GI local activation and pharmacokinetics of MMT3-72 and its active metabolite MMT3-72-M2 in vivo, mice were dosed orally with 10 mg/kg MMT3-72 and sacrificed to collect tissues at different time points from 0-24 hrs. As shown in FIG. 4A, high concentration (Cmax>50,000 ng/g) of the compound MMT3-72 was observed in the GT content, including stomach content, small intestine content, and colon content. However, MMT3-72 was not detected in the small intestine tissues, colon tissue, or systemic circulation. In contrast, high levels of the active metabolite MMT3-72-M2 were detected in the colon tissue and small intestinal tissues (Cmax>1500 ng/g) (FIG. 4B). At 24 hrs, the Cmin was 88.5 ng/mil in the small and large intestine tissues that was higher than the IC₅₀ in inhibiting JAK1, JAK2 and TYK2 relevant targets to treat UC. Conversely, MMT3-72-M2 concentration from 10-24 hrs was lower than IC₅₀ in inhibiting JAK3. Further, the concentration of MMT3-72-M2 in plasma is minimal (Cmax 8 ng/ml) and was undetectable after 4 hrs. Further, MMT3-72 was activated more to release MMT3-72-M2 in the colon than in the small intestine since MMT3-72-M2 in the colon content is 10-fold more than that in small intestine (FIG. 4C). These findings showed that (1) MMT3-72 was not absorbed into systemic circulation, was retained in the GI tract, and mainly activated in the colon region to release active metabolite MMT3972-M2, (2) the active metabolite MMT3972-M2 accumulated highly in the colon tissue and small intestinal tissues, which may inhibit JAK1, JAK2, and TYK2 for its therapeutic effects, (3) none of MMT3-72 and only low level of the active metabolite MMT3-72-M2 were detected in the systemic circulation, which has potential to avoid systemic toxicity of JAK inhibitions.

It is worth noting; that the design of MMT3-72 is distinctly different from the design of Izencitinib (TD-1473), which reduced absorption potential to limit systemic exposure but without a local-activation mechanism. The design of drugs (such as TD-1473) with only reduced absorption potential but without activation mechanism would reduce drug penetration in the colon tissue limiting its efficacy in human trials. In contrast, MMT3-72 was designed to not only reduce the GI absorption potential, but also have local activation properties to release active form of MMT3-72-M2 that can easily penetrate colon tissue to reach therapeutic concentration in the colon tissues while minimize drug exposure in the systemic circulation.

### MMT3-72 Exhibited Superior Efficacy in Treating UC in Mice.

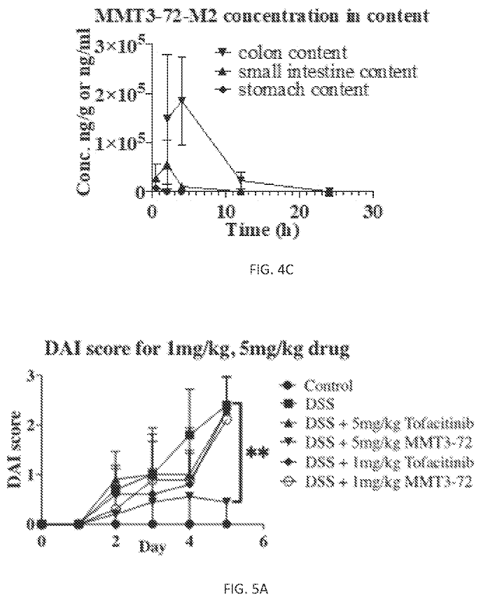

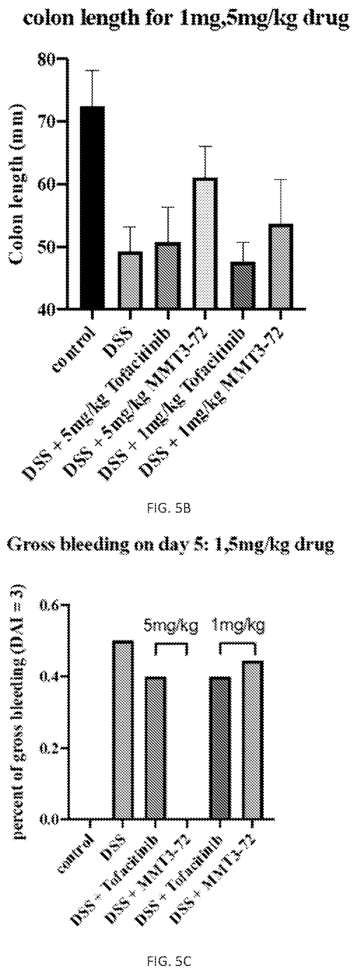

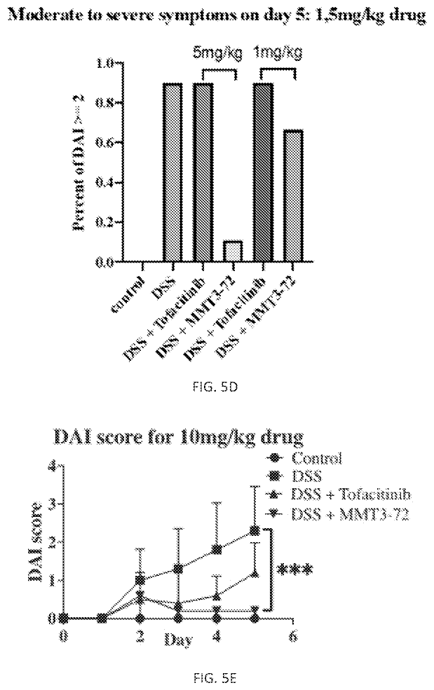

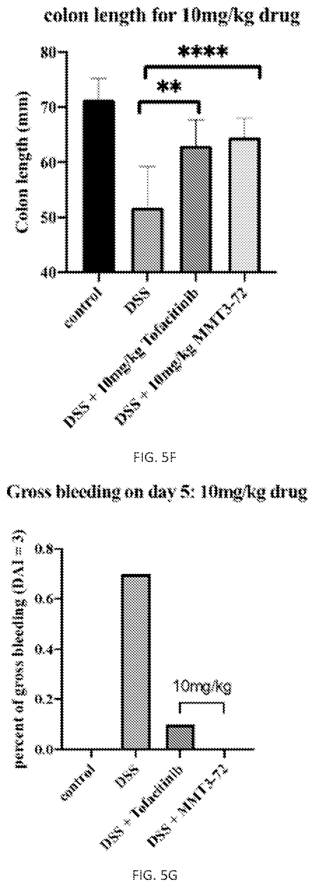

To evaluate the efficacy of MMT3-72 in the treatment of UC in vivo, a colitis model was established in mice using dextran sodium sulfate (DSS). DSS in drinking water could trigger colitis in mice. The DSS-induced colitis model is widely used because of its relatively easy administration and high similarity with human UC. In this study, mice treated with 3% DSS water developed symptoms of colitis such as bloody stools and diarrhea on day 5. Disease activity index (DAI) was monitored for the severeness of disease in mice: Normal stool consistency with negative hemoccult: score 0; Soft stools with positive hemoccult: score 1; Very soft stools with traces of blood: score 2; Watery stools with visible rectal bleeding: score 3. To evaluate the efficacy of MMT3-72 in comparison with FDA approved JAK inhibitor (Tofacitinib) for UC treatment, mice were treated orally with 1 mg/kg and 5 mg/kg of both drugs (FIGS. 5A, 5C, and 5D). MMT3-72 (5 mg/kg) improved DAT score by 5-fold in comparison with DSS induced colitis, while tofacitinib (5 mg/kg) did not show any improvement of DAI score (FIG. 5A). In MMT3-72 (5 mg/kg) treatment group, no mouse developed severe colitis and only 10% mice (n=10) developed moderate colitis (FIGS. 5C and 5D). In contrast, in Tofacitinib treatment group (5 mg/kg), 40% mice (n=10) developed severe colitis and 80% developed moderate colitis (FIG. 4C, 4D). Low doses (1 mug/kg) of MMT3-72 (1 mg/kg) and Tofacitinib (1 mg/kg) did not improve DAI score or disease severity in DSS-induced colitis (FIGS. 5A, 5C, and 5D).

High doses (10 mg/kg) of both MMT3-72 and tofacitinib were tested for treatment of DSS-induced UC (FIGS. 5E, 5G, and 5H). MMT3-72 (10 mg/kg) improved DAI score by 10-fold in DSS-induced colitis model, and no mice (n=10) developed moderate or severe colitis. In comparison, Tofacitinib (10 mg/kg) also showed improvement of DAI score, and only 10% of mice developed severe disease with gross bleeding, and only 20% of mice developed moderate colitis. High dose (10 mg/kg) of both MMT3-72 and tofacitinib recovered the colon length from DSS-induced colitis (FIG. 5F). These data suggest the MMT3-72 has advantages in the treatment of UC. 5.)

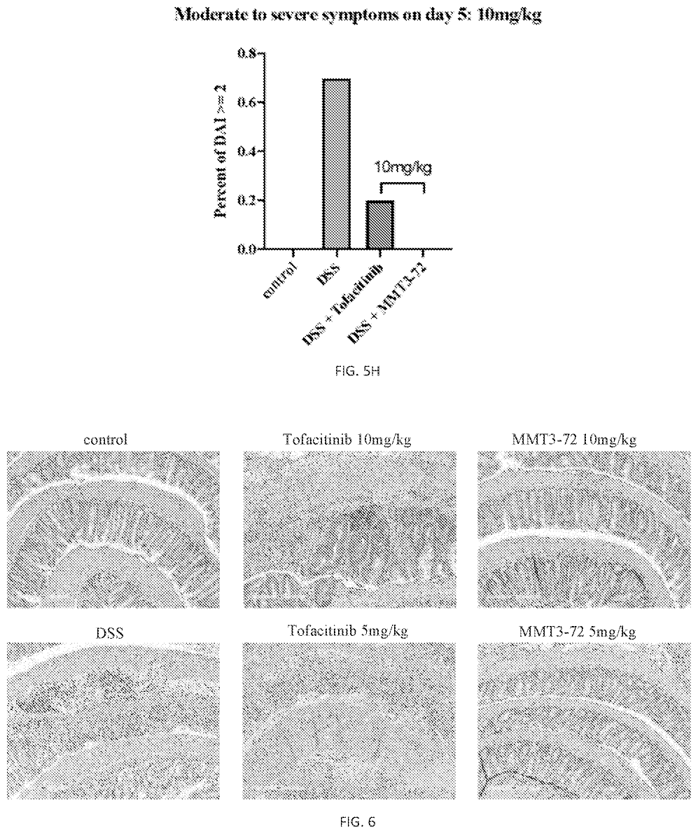

In order to further evaluate the efficacy of MMT3-72 in reducing colon inflammation and tissue injury, H&E staining of colon tissues from the above in vivo studies was performed as shown in FIG. 6. The DSS induced-colitis showed severe and diffuse destruction of the epithelial layer with extensive immune cell infiltration in the epithelium. MMT3972 (5, 10 mg/kg) reduced epithelial loss and decreased infiltration of immune cells in DSS induced colitis model. In contrast, tofacitinib (5 mg/kg) did not show improvement of epithelial cell loss and infiltration of immune cells in DSS-induced colitis model while tofacitinib (10 mg/kg) showed moderate improvement.

### Example 3

### JAK Inhibitors with Systemic Function for Treatment of Cancer and Autoimmune Diseases

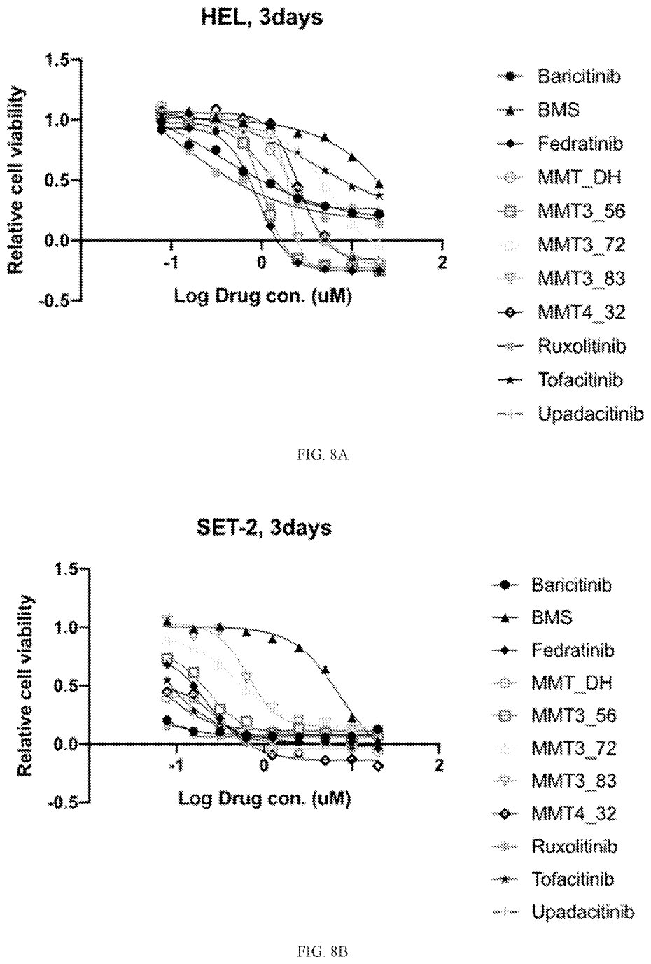

To assess MMT3-72 and MMT3-72-M2's effect of growth inhibition on the JAK related cell population, cytotoxicity experiments were performed on HEL and SET-2 cell lines using commercially available JAK inhibitors (FIGS. 8A and 8B). MMT3-72 did not have a strong inhibition on either cell lines while MMT3-72-M2 demonstrated better inhibition (Table 2).

TABLE 2
IC₅₀ of different JAK inhibitors in HEL cells and SET-2 cells

| Compound | HEL IC₅₀ (μM) | SET-2 IC₅₀ (μM) |
|---|---|---|
| Baricitinib | 0.3399 | ~0.007836 |
| Deucravacitinib | ~347.0 | 7.384 |
| Fedratinib | 0.9036 | 0.2097 |
| MMT3-56 | 1.031 | 0.2301 |
| MMT3-72 | 5.591 | 0.515 |
| MMT3-83 | 2.038 | 0.6448 |
| MMT3-72-M2 | 2.594 | 0.3316 |
| Ruxolitinib | 0.1139 | ~0.1452 |
| Tofacitinib | 3.693 | ~7.308e−6 |
| Upadacitinib | 1.009 | 0.06963 |

Cell Culture and anti-proliferative assay. Human cell lines HEL 92.1.7 and SET-2 were obtained from American Type Culture Collection (ATCC) and Leibniz Institute DSMZ-German Collection of Microorganisms and Cell Cultures GmbH, respectively. HEL and SET-2 cells were cultured in RMPI 1640 medium (Life Technologies Corporation, New York, USA) supplemented with 10% or 20% fetal bovine serum (FBS, Life Technologies Corporation, New York, USA), respectively. Cells were incubated at 37° C. in a humidified atmosphere of 5% CO₂. Cells were seeded into 96-well culture plates at the density of approximately 8000 cells per well and treated with various concentrations of compounds for 3 days in final volumes of 200 μL. Upon end point, cells were treated using CellTiter 96 Aqueous Assay Reagents (Promega Corporation, Madison, USA) according to the provider's instruction. Briefly, MITS and PMS solution were thawed and mixed (20:1, v/v) before use and pipet appropriate amount of the mixed solution into each well of the 96 well assay plates. The plates were incubated for 1-4 hours at 37° C. in a humidified atmosphere of 5% CO₂. The absorbance at 490 nm was recorded using CYTATION 5 imaging reader (BioTek, VT, USA). IC₅₀ values were calculated using percentage of growth versus untreated control.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A Janus kinase (JAK) inhibitor analog, or a pharmaceutically acceptable salt thereof, wherein the JAK inhibitor analog has the structure:
A-L-B
wherein:
A is a JAK inhibitor moiety;
L is a cleavable linker; and
B is a prodrug moiety.

2. The JAK inhibitor analog of claim 1, or a pharmaceutically acceptable salt thereof, wherein the JAK inhibitor moiety is derived from abrocitinib, baricitinib, cerdulatinib, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

3. The JAK inhibitor analog of claim 1 or claim 2, or a pharmaceutically acceptable salt thereof, wherein the JAK inhibitor moiety comprises a benzimidazole moiety, a pyrrolopyrimidine moiety, or a biaryl meta-pyrimidine moiety.

4. The JAK inhibitor analog of any of claims 1-3, or a pharmaceutically acceptable salt thereof, wherein the cleavable linker comprises at least one selectively cleavable group or bond.

5. The JAK inhibitor analog of claim 4, or a pharmaceutically acceptable salt thereof, wherein the selectively cleavable group or bond is enzymatically cleavable.

6. The JAK inhibitor analog of any of claims 1-5, or a pharmaceutically acceptable salt thereof, wherein the cleavable linker comprises an azo group.

7. The JAK inhibitor analog of any of claims 1-6, or a pharmaceutically acceptable salt thereof, wherein
L comprises 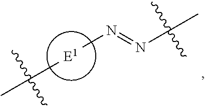
wherein:
E¹ is a C₄-C₁₀ cycloalkylene, C₄-C₁₀ heterocyclylene, C₄-C₁₀ arylene or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, amino-C₁-C₆-alkyl, or —COO—R^{1a}; and
R^{1a} is hydrogen or C₁-C₆ alkyl.

8. The JAK inhibitor analog of claim 7, or a pharmaceutically acceptable salt thereof, wherein E¹ is a C₄-C₁₀ arylene or C₄-C₁₀ heteroarylene, optionally substituted with 1 or 2 substituents independently selected from C₁-C₆ alkyl, amino, C₁-C₆-alkoxy, hydroxy, hydroxy-C₁-C₆-alkyl, amino-C₁-C₆-alkyl, or —COO—R^{1a}.

9. The JAK inhibitor analog of any of claims 1-8, or a pharmaceutically acceptable salt thereof, wherein L comprises

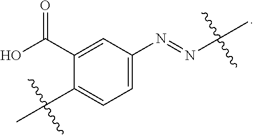 [SMILES: */N=N/c1ccc(*)c(C(=O)O)c1]

10. The JAK inhibitor analog of any of claims 7-9, or a pharmaceutically acceptable salt thereof, wherein L further comprises a combination of one or more groups selected from —CH₂—, —O—, —NR^{1b}—, arylene and heteroarylene and R^{1b} is hydrogen or C₁-C₆ alkyl.

11. The JAK inhibitor analog of any of claims 7-10, or a pharmaceutically acceptable salt thereof, wherein L further comprises

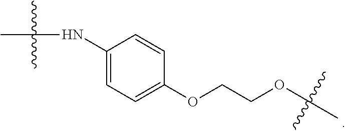 [SMILES: *Nc1ccc(OCCO*)cc1]

12. The JAK inhibitor analog of any of claims 1-11, or a pharmaceutically acceptable salt thereof, wherein
B comprises 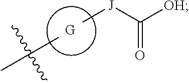
wherein:
G is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, or amino-C₁-C₆-alkyl; and
J is a bond or a linker comprising a combination of one or more groups selected from —C(R^{1c})₂—, —CH═CH—, —C≡C—, —O—, —NR^{1c}—, —S—, —C(O)—, —C(NR^{1c})—, —S(O)—, and —S(O)₂—, wherein each R^{1c} is independently selected from hydrogen, C₁-C₆ alkyl, C₂-C₆ alkenyl, and C₂-C₆ alkynyl.

13. The JAK inhibitor analog of claim 12, or a pharmaceutically acceptable salt thereof, wherein J is a linker comprising a combination of one or more groups selected from —C(R^{1c})₂—, —NR^{1c}—, and —C(O)—, wherein each R^{1c} is independently selected from hydrogen and C₁-C₆ alkyl.

14. The JAK inhibitor analog of claim 12 or claim 13, or a pharmaceutically acceptable salt thereof, wherein J comprises

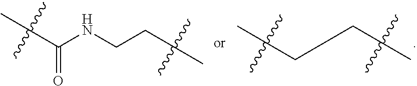 [SMILES: *CCNC(*)=O ; *CCC*]

15. The JAK inhibitor analog of claim 12, or a pharmaceutically acceptable salt thereof, wherein J is a bond.

16. The JAK inhibitor analog of any of claims 1-15, or a pharmaceutically acceptable salt thereof, wherein B comprises

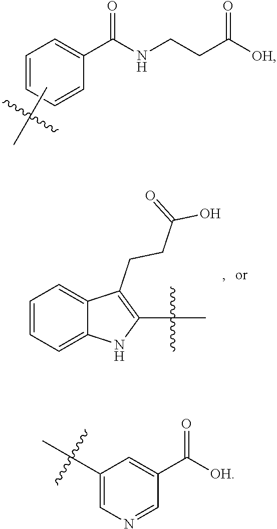

17. The JAK inhibitor analog of any of claims 1-16, wherein the JAK1 inhibitor analog is a compound of formula (I): 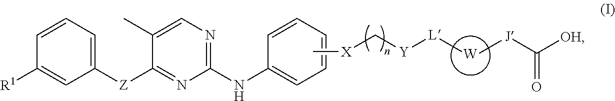
or a pharmaceutically acceptable salt thereof, wherein:
Z is NR^{a}, wherein R^{a} is H or C₁-C₆ alkyl;
R is alkyl or SO₂—R², wherein R² is selected from C₁-C₆ alkyl, C₃-C₉ cycloalkyl, C₃-C₉ heterocycle, and N(R^{b})₂, and wherein each R^{b} is independently selected from hydrogen, C₁-C₆ alkyl, C₃-C₉ cycloalkyl, and C₃-C₉ heterocycle, or both R^{b} are taken together with the nitrogen atom to which they are attached to form an optionally substituted 5- or 6-membered ring;
X is O, SO₂, or CH₂;
Y is NH, O, or CH₂;
W is a C₄-C₁₀ cycloalkylene, C₄-C₁₀ heterocyclylene, C₄-C₁₆ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, or amino-C₁-C₆-alkyl;
J′ is a bond or a linker comprising a combination of one or more groups selected from —C(R^{c})₂—, —CH═CH—, —C≡C—, —O—, —NR^{c}—, —S—, —C(O)—, —C(NR^{c})—, —S(O)—, and —S(O)₂—, wherein each R^{c} is independently selected from hydrogen, C₁-C₆ alkyl, C₂-C₆ alkenyl, or C₂-C₆ alkynyl;
n is 1, 2, 3, 4, 5, or 6; and
L′ is cleavable linker.

18. The JAK inhibitor analog of claim 17, or a pharmaceutically acceptable salt thereof, wherein J′ is a linker comprising a combination of one or more groups selected from —C(R^{c})₂—, —NR^{c}—, and —C(O)—, wherein each R^{c} is independently selected from hydrogen and C₁-C₆ alkyl.

19. The JAK inhibitor analog of claim 17 or claim 18, or a pharmaceutically acceptable salt thereof, wherein J′ comprises

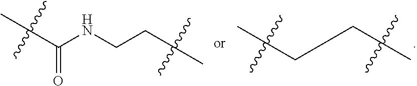 [SMILES: *CCNC(*)=O ; *CCC*]

20. The JAK inhibitor analog of claim 17, or a pharmaceutically acceptable salt thereof wherein J′ is a bond.

21. The JAK inhibitor analog of any of claims 1-20, wherein the JAK inhibitor analog is a compound of formula (Ia): 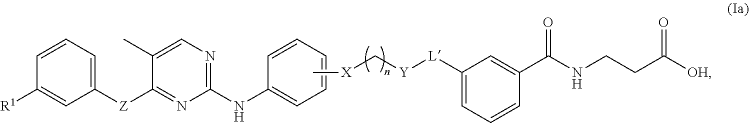
or a pharmaceutically acceptable salt thereof.

22. The JAK inhibitor analog of any of claims 17-21, or a pharmaceutically acceptable salt thereof, wherein Z is NH.

23. The JAK inhibitor analog of any of claims 17-22, or a pharmaceutically acceptable salt thereof, wherein R¹ is —SO₂—N(R^{b})₂.

24. The JAK inhibitor analog of claim 23, or a pharmaceutically acceptable salt thereof, wherein one R^{b} is hydrogen and the other is C₁-C₆ alkyl.

25. The JAK inhibitor analog of any of claims 17-24, or a pharmaceutically acceptable salt thereof, wherein X and Y are O.

26. The JAK inhibitor analog of any of claims 17-25, or a pharmaceutically acceptable salt thereof, wherein n is 1, 2, or 3.

27. The JAK inhibitor analog of any of claims 17-26, or a pharmaceutically acceptable salt thereof, wherein
L′ comprises 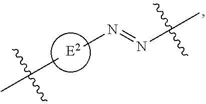
E² is a C₄-C₁₀ cycloalkylene, C₄-C₁₀ heterocyclylene, C₄-C₁₀ arylene, or C₄-C₁₀ heteroarylene, wherein each cycloalkylene, heterocyclylene, arylene, or heteroarylene is optionally substituted with 1, 2, 3, or 4 substituents independently selected from C₁-C₆ alkyl, amino, C₁-C₆-alkoxy, hydroxy, hydroxy-C₁-C₆-alkyl, amino-C₁-C₆-alkyl, or —COO—R^{d}; and
R^{d} is hydrogen or C₁-C₆ alkyl.

28. The JAK inhibitor analog of claim 27, or a pharmaceutically acceptable salt thereof, wherein E² is a C₄-C₁₀ arylene or C₄-C₁₀ heteroarylene, optionally substituted with 1 or 2 substituents independently selected from C₁-C₆ alkyl, amino, C₁-C₆-alkoxy, hydroxy, hydroxy-C₁-C₆-alkyl, amino-C₁-C₆-alkyl, or —COO—R^{d}.

29. The JAK inhibitor analog of any of claims 17-28, or a pharmaceutically acceptable salt thereof, wherein L′ comprises

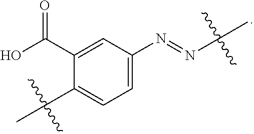 [SMILES: */N=N/c1ccc(*)c(C(=O)O)c1]

30. The JAK inhibitor analog of any of claims 27-29, or a pharmaceutically acceptable salt thereof, wherein L′ further comprises a combination of one or more groups selected from —CH₂—, —O—, —NR—, arylene and heteroarylene.

31. The JAK inhibitor analog ofany of claims 27-30, or a pharmaceutically acceptable salt thereof, wherein L′ further comprises

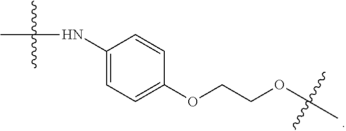 [SMILES: *Nc1ccc(OCCO*)cc1]

32. The JAK inhibitor analog of any of claims 1-31, wherein the compound is: 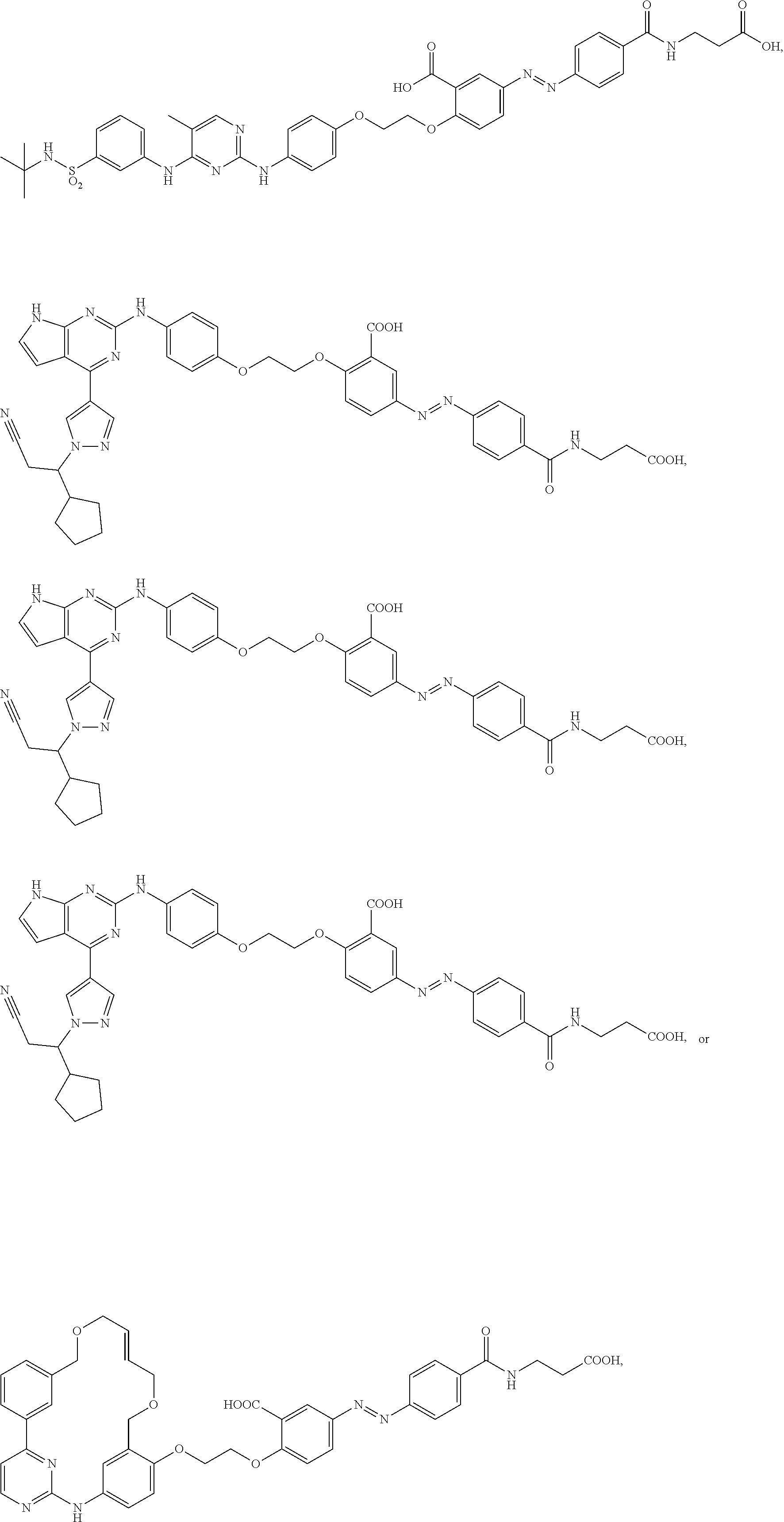
or a pharmaceutically acceptable salt thereof.

33. A pharmaceutical composition comprising an effective amount of a JAK inhibitor analog of any one of claims 1-32, or a pharmaceutically acceptable salt thereof, and a pharmaceutically acceptable carrier.

34. A method of treating or preventing a disease or disorder comprising administering an effective amount of a JAK inhibitor analog of any one of claims 1-32, or a pharmaceutically acceptable salt thereof, or a pharmaceutical composition of claim 33, to a subject in need thereof.

35. The method of claim 34, wherein the disease or disorder is cancer, an autoimmune disease, or an inflammatory disease.

36. The method of claim 34 or claim 35, wherein the disease or disorder is a gastrointestinal inflammatory disease or disorder.

37. The method of any of claims 34-36, wherein the disease or disorder is inflammatory bowel disease.

38. The method of claim 37, wherein the inflammatory bowel disease is ulcerative colitis or Crohn's disease.

39. The method of claim 34 or claim 35, wherein the disease or disorder is cancer.

40. The method of claim 39, wherein the subject has cancer, has had cancer, is predisposed to cancer, or has a family history of cancer.

41. The method of any of claim 34-40, wherein the JAK inhibitor analog, or a pharmaceutically acceptable salt or composition thereof is administered orally.

42. A compound of formula (II): 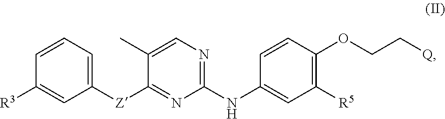
or a pharmaceutically acceptable salt thereof, wherein:
Q is 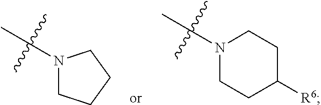
Z′ is NR^{c}, wherein R^{c} is H or C₁-C₆ alkyl;
R³ is alkyl or SO₂—R⁴, wherein R⁴ is selected from C₁-C₆ alkyl, C₃-C₉ cycloalkyl, C₃-C₉ heterocycle, and N(R^{d})₂, and wherein each R^{d} is independently selected from hydrogen, C₁-C₆ alkyl, C₃-C₉ cycloalkyl, and C₃-C₉ heterocycle, or both R^{d} are taken together with the nitrogen atom to which they are attached to form an optionally substituted 5- or 6-membered ring;
R⁵ is hydrogen, —CH₂—OCH₃ or —CH₂—(OCH₂CH₂)—OCH₃; and
R⁶ is —OCH₃ or —OCH₂CH₂—OCH₃.

43. The compound of claim 42, wherein Z′ is NH.

44. The compound of claim 42 or 43, wherein R³ is SO₂—N(R^{d})₂.

45. The compound of claim 44, wherein one R^{d} is hydrogen and one R^{d} is C₁-C₆ alkyl.

46. The compound of any of claims 42-45, wherein Q is 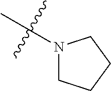 [SMILES: *N1CCCC1]
and R⁵ is —CH₂—OCH₃ or —CH₂—(OCH₂CH₂)—OCH₃.

47. The compound of any of claims 42-45, wherein Q is 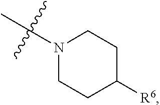
R⁵ is hydrogen, and R⁶ is —OCH₃ or —OCH₂CH₂—OCH₃.

48. The compound of any of claims 42-45, wherein Q is 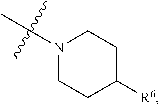
R⁵ is —CH₂—OCH₃, and R⁶ is —OCH₃ or —OCH₂CH₂—OCH₃.

49. The compound of any of claims 42-45, wherein Q is 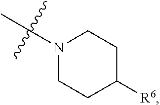
R⁵ is —CH₂—(OCH₂CH₂)—OCH₃, and R⁶ is —OCH₃— or —OCH₂CH₂—OCH₃.

50. A pharmaceutical composition comprising an effective amount of a compound of any one of claims 42-49, or a pharmaceutically acceptable salt thereof, and a pharmaceutically acceptable carrier.

51. A method of treating or preventing a disease or disorder comprising administering an effective amount of a compound of any one of claims 42-49, or a pharmaceutically acceptable salt thereof, or a In another aspect, disclosed herein is a composition of claim 50, to a subject in need thereof.
